(19) United States
(12) Patent Application Publication
(10) Pub. No.: US 2021/0054367 A1
(43) Pub. Date: Feb. 25, 2021

(54) METHODS AND COMPOSITIONS FOR TARGETED EDITING OF POLYNUCLEOTIDES

(71) Applicant: SYNGENTA PARTICIPATIONS AG, Basel (CH)

(72) Inventors: Jiang Li, Beijing (CN); Jianping Xu, Beijing (CN); Shon Li, Beijing (CN); Lizhao Geng, Beijing (CN)

(73) Assignee: SYNGENTA PARTICIPATIONS AG, Basel (CH)

(21) Appl. No.: 17/052,210

(22) PCT Filed: Apr. 30, 2019
(86) PCT No.: PCT/US2019/029891

§ 371 Date: Nov. 2, 2020

(30) Foreign Application Priority Data
May 10, 2018 (CN) PCT/CN2018/086365

(51) Int. Cl. C12N 15/10; C12N 15/113

(52) U.S. Cl. CPC C12N 15/1082; C12N 15/113

(57) ABSTRACT

The present invention relates to methods and compositions for modifying a target site in a DNA molecule. The present disclosure provides a DNA-targeting RNA duplex which comprises a crRNA molecule and a tracrRNA molecule and methods of using these molecules for modification of a target DNA. Modifications include targeted transgene insertion, targeted allelic replacement, and targeted mutagenesis.

### CROSS REFERENCE TO RELATED APPLICATIONS

This application claims the benefit of U.S. Provisional Patent Application No. PCT/CN2018/086365 filed May 10, 2018, which is incorporated by reference in its entirety.

### SEQUENCE LISTING

A Sequence Listing in ASCII text format, submitted under 37 C.F.R. § 1.821, entitled “81555_ST25.txt”, 82 kilobytes in size, generated on Apr. 24, 2018, is provided in lieu of a paper copy. This Sequence Listing is hereby incorporated by reference into the specification for its disclosures.

### FIELD OF THE INVENTION

The present invention relates to methods and compositions for targeted transgene insertion, targeted allelic replacement, or targeted mutagenesis in the genome of a cell.

### BACKGROUND OF THE INVENTION

Recent advances in the field of targeted genomic modifications have made it so that routine targeted modifications may soon be possible. Significant advances have been made in the last few years towards the development of methods and compositions to target and cleave genomic DNA by site-specific nucleases, for example Zinc Finger Nucleases (ZFNs), meganucleases, Transcription Activator-Like Effector Nucleases (TALENS) and Clustered Regularly Interspaced Short Palindromic Repeats/CRISPR-associated nuclease (CRISPR/Cas), which act in complex with either an engineered crRNA-tracrRNA duplex or with a single guide RNA. These site-specific nucleases can induce targeted mutagenesis, induce targeted deletions of DNA sequences, and facilitate targeted recombination of an exogenous donor DNA polynucleotide, such as a transgene, within a targeted DNA sequence.

In the type II CRISPR system, the Cas9 nuclease, guided by a dual-guide system comprising a crRNA:tracrRNA duplex, is sufficient to cleave the target DNA (Jinek et al., 2012, Science: 337: 816-821). Site-specific cleavage occurs at locations determined by base-pairing complementarity between the crRNA and the target DNA and a short motif, referred to as the protospacer adjacent motif (PAM), juxtaposed to the complementary region in the target DNA. The crRNA alone cannot direct Cas9 to the target DNA; the tracrRNA, which pairs with sequences of the crRNA, is required to form a protein-binding segment that enables complex formation between the crRNA-tracrRNA duplex and the Cas9 enzyme. However, it is not known if the interactions between the crRNA and the tracrRNA can be optimized to increase Cas9 targeting and/or mutagenesis efficiency.

### SUMMARY OF THE INVENTION

The present invention provides a DNA-targeting RNA duplex which comprises a crRNA molecule and its corresponding tracrRNA molecule, wherein the crRNA molecule and the tracrRNA molecule comprise the nucleic acid sequences of, respectively, SEQ ID NO: 55 and 56, SEQ ID NO: 57 and 58, SEQ ID NO: 59 and 60, SEQ ID NO: 61 and 62, SEQ ID NO: 63 and 64, SEQ ID NO: 65 and 66, SEQ ID NO: 67 and 68, SEQ ID NO: 69 and 70, SEQ ID NO: 71 and 72, SEQ ID NO: 73 and 74, SEQ ID NO: 75 and 76, SEQ ID NO: 77 and 78, SEQ ID NO: 79 and 80, SEQ ID NO: 81 and 82, or SEQ ID NO: 83 and 84, wherein the crRNA further comprises a DNA-targeting segment which comprises a nucleic acid sequence that is complementary to a sequence in a target DNA molecule, whereby the DNA-targeted RNA duplex targets and hybridizes with the target DNA sequence. The crRNA and corresponding tracrRNA molecules of the invention are engineered, meaning that they were created by the hand of man and are not naturally occurring.

The present invention also provides a nucleic acid molecule comprising a nucleic acid sequence encoding at least one crRNA and/or at least one tracrRNA of the invention. The nucleic acid molecule may encode for more than one crRNA molecule, wherein the multiple crRNA molecules have different protospacer sequences. Alternatively, the nucleic acid molecule may encode for multiple crRNA molecules which have the same protospacer sequence. The nucleic acid molecule may also encode for multiple tracrRNA molecules, or it may encode for a single tracrRNA molecule multiple times. The nucleic acid molecule may be a DNA or an RNA molecule. In some embodiments, the nucleic acid molecule is circularized. In other embodiments, the nucleic acid molecule is linear. In some embodiments, the nucleic acid molecule is single stranded, partially double-stranded, or double-stranded.

The present invention also provides an engineered, non-naturally occurring system for targeted mutagenesis comprising a DNA-targeting RNA duplex of the invention and a site-directed modifying polypeptide, wherein the DNA-targeting RNA duplex comprises a crRNA molecule and its corresponding tracrRNA molecule, wherein the crRNA molecule and the tracrRNA molecule comprise the nucleic acid sequences of, respectively, SEQ ID NO: 55 and 56, SEQ ID NO: 57 and 58, SEQ ID NO: 59 and 60, SEQ ID NO: 61 and 62, SEQ ID NO: 63 and 64, SEQ ID NO: 65 and 66, SEQ ID NO: 67 and 68, SEQ ID NO: 69 and 70, SEQ ID NO: 71 and 72, SEQ ID NO: 73 and 74, SEQ ID NO: 75 and 76, SEQ ID NO: 77 and 78, SEQ ID NO: 79 and 80, SEQ ID NO: 81 and 82, or SEQ ID NO: 83 and 84, wherein the crRNA further comprises a nucleic acid sequence that is complementary to a sequence in a target DNA molecule, whereby the crRNA-tracrRNA dual guide complex targets and hybridizes with the target DNA sequence, and the site-directed modifying polypeptide cleaves the DNA molecule. The crRNA molecule of the invention further comprises a protospacer sequence, which is the DNA-targeting segment of the crRNA molecule of the invention and is complementary to a sequence in a target DNA molecule.

In some embodiments, the crRNA molecule, its corresponding tracrRNA molecule, and the site-directed modifying polypeptide are encoded within at least one nucleic acid molecule, wherein the crRNA molecule and the tracrRNA molecule are encoded by nucleic acid sequences comprising, respectively, SEQ ID NO: 3 and 4, SEQ ID NO: 5 and 6, SEQ ID NO: 7 and 8, SEQ ID NO: 9 and 10, SEQ ID NO: 11 and 12, SEQ ID NO: 13 and 14, SEQ ID NO: 15 and 16, SEQ ID NO: 17 and 18, SEQ ID NO: 19 and 20, SEQ ID NO: 21 and 22, SEQ ID NO: 23 and 24, SEQ ID NO: 28 and 29, SEQ ID NO: 30 and 31, SEQ ID NO: 32 and 33, or SEQ ID NO: 34 and 35, or the complements thereof, wherein the crRNA further comprises a DNA-targeting segment which comprises a nucleic acid sequence that is complementary to a sequence in a target DNA molecule, whereby the crRNA-tracrRNA dual guide complex targets and hybridizes with the target DNA sequence, and the site-directed modifying polypeptide cleaves the DNA molecule.

In some embodiments, the nucleic acid molecule on which the crRNA and/or tracrRNA is encoded is a vector. In further embodiments, the nucleic acid molecule is a vector capable of transformation, for example biolistic transformation, Agrobacterium-mediated transformation, or PEG/electroporation transformation. In some embodiments, the site-directed modifying polypeptide is encoded on the same nucleic acid molecule on which the crRNA and tracrRNA molecules are encoded. In other embodiments, the site-directed modifying polypeptide is encoded on a different nucleic acid molecule from that which encodes the crRNA and tracrRNA molecules. In some embodiments, the crRNA and the tracrRNA molecules are encoded in different expression cassettes. In other embodiments, the crRNA and the tracrRNA molecules are encoded in the same expression cassette.

The present invention also provides a RNA molecule comprising at least one crRNA segment and at least one of its corresponding tracrRNA segment, wherein the segments are operably linked at the 5′ and/or 3′ end to a tRNA cleavage sequence. In some embodiments, the RNA molecule may be present in a cell capable of tRNA cleavage. Following tRNA cleavage, the crRNA segment becomes a crRNA molecule of the invention, and the tracrRNA segment becomes a tracrRNA molecule of the invention, so that the crRNA and tracrRNA molecules are separate and distinct molecules which are capable of forming a DNA-targeting RNA duplex. In some embodiments, the RNA molecule comprises a tRNA-crRNA-tRNA-tracrRNA or a tRNA-tracrRNA-tRNA-crRNA in tandem alignment. In some embodiments, at least one of the resulting crRNA molecules and its corresponding tracrRNA molecule comprise the nucleic acid sequences of, respectively, SEQ ID NO: 55 and 56, SEQ ID NO: 57 and 58, SEQ ID NO: 59 and 60, SEQ ID NO: 61 and 62, SEQ ID NO: 63 and 64, SEQ ID NO: 65 and 66, SEQ ID NO: 67 and 68, SEQ ID NO: 69 and 70, SEQ ID NO: 71 and 72, SEQ ID NO: 73 and 74, SEQ ID NO: 75 and 76, SEQ ID NO: 77 and 78, SEQ ID NO: 79 and 80, SEQ ID NO: 81 and 82, or SEQ ID NO: 83 and 84, wherein the crRNA further comprises a nucleic acid sequence that is complementary to a sequence in a target DNA sequence.

The present invention also provides a nucleic acid molecule comprising at least one expression cassette which expresses the RNA molecule comprising tRNA cleavage sites described herein. This nucleic acid molecule is an improved construct for delivery of crRNA and tracrRNA molecules to a cell and to a target DNA molecule. The nucleic acid molecule may be present in a cell capable of tRNA cleavage. In some embodiments, a crRNA molecule of the invention, the corresponding tracrRNA molecule of the invention, and at least two tRNA cleavage sequences are encoded within the same expression cassette, whereby following tRNA cleavage the crRNA and the tracrRNA molecules are separate and distinct molecules.

In some embodiments, the nucleic acid molecule which expresses an RNA molecule comprising at least one tRNA cleavage site comprises at least one expression cassette which comprises a promoter driven by a RNA polymerase II. In further embodiments, the promoter driven by a RNA polymerase II is at least 90% identical to SEQ ID NO: 85. In some embodiments, the nucleic acid molecule comprises at least one expression cassette which comprises a promoter driven by a RNA polymerase III. In further embodiments, the promoter driven by a RNA polymerase III is at least 90% identical to SEQ ID NO: 86. In some embodiments, the nucleic acid molecule of the invention comprises at least two expression cassettes, one of which comprises a promoter driven by a RNA polymerase II and the other of which comprises a promoter driven by a RNA polymerase III. In further embodiments, the first expression cassette comprises a promoter at least 90% identical to SEQ ID NO: 85 and the second expression cassette comprises a promoter at least 90% identical to SEQ ID NO: 86.

In some embodiments, the nucleic acid molecule described directly above comprises at least one expression cassette, wherein the nucleic acid sequence of the expression cassette is any of SEQ ID NOs: 87-94. The twenty N's within SEQ ID NOs: 87-94 represent the protospacer of the crRNA molecules encoded within the expression cassette. As described herein, the protospacer sequence may be at least 12 nucleotides in length, with at least 90%, at least 91%, at least 92%, at least 93%, at least 94%, at least 95%, at least 96%, at least 97%, at least 98%, at least 99%, or 100% complementarity to the target sequence of the target DNA molecule.

The present invention also provides a method of site-specific modification of a target DNA, the method comprising contacting the target DNA with: (i) a DNA-targeting RNA duplex, or a DNA molecule encoding the same, wherein the DNA-targeting RNA duplex is a DNA-targeting RNA duplex of the invention as described herein, and (ii) a site-directed modifying polypeptide, or a DNA molecule encoding the same, wherein the site-directed modifying polypeptide comprises an RNA-binding portion that interacts with the DNA-targeting RNA, and an activity portion that exhibits site-directed enzymatic activity.

Methods of the invention include site-specific modification of a target DNA wherein the DNA-modifying enzymatic activity is nuclease activity. The nuclease may introduce a single-strand or a double-stranded break in the target DNA. The DNA-targeting RNA duplex and/or the site-directed modifying polypeptide may contact the target DNA under conditions that are permissive for nonhomologous end joining (NHEJ) or homology-directed repair. In some embodiments, the target DNA may be modified as a result of the repair process, and not as a direct result of the enzymatic activity of the site-directed modifying polypeptide which may act only as a site-directed nuclease.

The present invention also provides a method of site-specific modification wherein the target site is modified by the insertion of a nucleic acid sequence. This sequence is provided by a donor molecule. In this method, the target DNA is contacted with: (i) a DNA-targeting RNA duplex, or a DNA molecule encoding the same, wherein the DNA-targeting RNA duplex is a DNA-targeting RNA duplex of the invention as described herein; (ii) a site-directed modifying polypeptide, or a DNA molecule encoding the same, wherein the site-directed modifying polypeptide comprises an RNA-binding portion that interacts with the DNA-targeting RNA, and an activity portion that exhibits site-directed enzymatic activity; and (iii) a donor polynucleotide, wherein the donor polynucleotide, a portion of the donor polynucleotide, a copy of the donor polynucleotide, or a portion of a copy of the donor polynucleotide integrates into the target DNA.

In some embodiments of the methods of the invention, DNA molecules encoding for the DNA-targeting RNA duplex and/or site-directed modifying polypeptide are introduced or delivered into the cell comprising the target DNA. In some embodiments, the DNA-targeting RNA duplex and the site-directed modifying polypeptide are encoded on the same DNA molecule. In other embodiments, they are encoded on separate DNA molecules. In further embodiments, the DNA molecule(s) are introduced into the cell by biolistic bombardment, Agrobacterium mediated-transformation, or any other methods known in the art. In some embodiments, the DNA molecules are transiently expressed, and do not incorporate into the genome of the cell. In some embodiments, the DNA molecules are stably transformed, and incorporate into the genome of the cell.

The present invention also provides a method of producing a plant, plant part, or progeny thereof comprising a site-specific modification of a target DNA, said method comprising regenerating a plant from a plant cell whose DNA has been modified by any of the methods of the invention described above. The present invention further provides the plant, plant part, or progeny thereof comprising a modification of its DNA which was produced by these methods.

### BRIEF DESCRIPTION OF THE FIGURES

FIG. 1 (FIG. 1) depicts the crRNA-tracrRNA duplex and indicates mutations d2 through d9 and d11 of the invention. The sequence of the crRNA is SEQ ID NO: 113. The sequence of the tracrRNA is SEQ ID NO: 114. d2 through d7 and d11 indicate the location of point mutations to the crRNA and the corresponding location in the tracrRNA to preserve the basepairing. Without the protospacer, the sequences of the crRNA for d2 and its corresponding tracrRNA are SEQ ID NO: 57-58; SEQ ID NO: 59-60 for d3, SEQ ID NO: 61-62 for d4, SEQ ID NO: 63-64 for d5, SEQ ID NO: 65-66 for d6, SEQ ID NO: 67-68 for d7, and SEQ ID NO: 75-76 for d11. d8 is a 9 nt addition which extends the crRNA at the 3′end to provide complementary basepairing with the 5′end of the tracrRNA, and the sequences of the mutated crRNA (without the protospacer) and the corresponding tracrRNA are SEQ ID NO: 69-70. d9 is a 8 nt deletion of the 5′ end of tracrRNA to eliminate the 5′ overhang of the tracrRNA, and the sequence of a crRNA for d9 (without the protospacer) and the mutated tracrRNA are SEQ ID NO: 71-72.

### BRIEF DESCRIPTION OF THE SEQUENCES IN THE SEQUENCE LISTING

SEQ ID NOs: 1-2 are DNA sequences encoding part of a crRNA and corresponding tracrRNA molecules. The crRNA sequence here does not include the DNA-targeting segment of the crRNA.

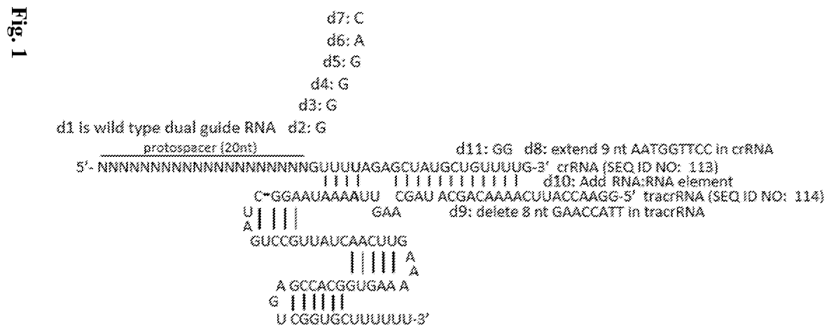

SEQ ID NOs: 3-24 are DNA sequences encoding part of engineered crRNAs and corresponding tracrRNA molecules. The crRNA sequences here do not include the DNA-targeting segment of the crRNA. These pairs are identified as mutations d1-d11 in FIG. 1 and in Table 1.

SEQ ID NO: 25 is a DNA sequence of an expression cassette encoding a tRNA-crRNA-tRNA-tracrRNA molecule, driven by a prOsU3 promoter, which is driven by a RNA polymerase III, and terminating at its 3′ end with a synthetic polyT terminator. This expression cassette is in vector 23999, as described in Example 2 and Table 1.

SEQ ID NO: 26 is a DNA sequence of an expression cassette encoding a tRNA-tracrRNA-tRNA-crRNA molecule, driven by a prOsU3 promoter, which is driven by a RNA polymerase III, and terminating at its 3′ end with a synthetic polyT terminator. This expression cassette is in vector 24000, as described in Example 2 and Table 1.

SEQ ID NO: 27 is a DNA sequence of an expression cassette encoding a single-guide RNA molecule, driven by a prOsU3 promoter, which is driven by a RNA polymerase III, and terminating at its 3′ end with a synthetic polyT terminator. This expression cassette is in vector 23127. The DNA-targeted segment of this sgRNA is the same DNA-targeting segment as in the engineered crRNA molecules, as described in Example 2 and Table 1.

SEQ ID NOs: 28-35 are DNA sequences encoding part of engineered crRNAs and its corresponding tracrRNA molecules, as described in Examples 3-4 and Table 2. The crRNA sequences here do not include the DNA-targeting segment of the crRNA.

SEQ ID NO: 36 is a DNA sequence comprising a wheat dwarf virus (WDV) DNA replicon, so that the engineered d9+d11 crRNA and the tracrRNA molecules are expressed from the WDV replicon.

SEQ ID NO: 37 is a DNA sequence of an expression cassette, driven by a prOsU3 promoter and terminated by a synthetic polyT terminator, which encodes an RNA molecule which comprises tRNA-tracrRNA-tRNA-crRNA-tRNA-tracrRNA-tRNA-crRNA, as described in Example 3.

SEQ ID NO: 38 is a DNA sequence of an expression cassette, driven by a prOsU3 promoter and terminated by an Arabidopsis thaliana terminator, which encodes an RNA molecule which comprises tRNA-tracrRNA-tRNA-crRNA-tRNA-tracrRNA-tRNA-crRNA, wherein the crRNA and tracrRNA comprise the d9 mutation, as described in Example 3.

SEQ ID NO: 39 is an amino acid sequence of the Cas9 variant used in the Examples.

SEQ ID NO: 40 is a DNA sequence of an expression cassette, driven by a prSoUbi promoter, which is driven by a RNA polymerase II, and terminated by an Agrobacterium tumefaciens terminator, which encodes an RNA molecule which comprises tRNA-tracrRNA-tRNA-crRNA-tRNA-tracrRNA-tRNA-crRNA-tRNA, as described in Example 3.

SEQ ID NO: 41 is a DNA sequence of an expression cassette, driven by a prOsU3 promoter and terminated by an A. thaliana terminator, which encodes an RNA molecule which comprises tRNA-tracrRNA-tRNA-crRNA-tRNA-tracrRNA-tRNA-crRNA, wherein the crRNA and tracrRNA comprise the d9 mutation, as described in Example 3.

SEQ ID NO: 42 is a DNA sequence of an expression cassette, driven by a prSoUbi promoter and terminated by an A. tumefaciens terminator, which encodes an RNA molecule which comprises tRNA-tracrRNA-tRNA-crRNA-tRNA-tracrRNA-tRNA-crRNA-tRNA, wherein the crRNA and tracrRNA comprise the d9 mutation, as described in Example 3.

SEQ ID NO: 43 is a DNA sequence of an expression cassette, driven by a prOsU3 promoter and terminated by a synthetic polyT terminator, which encodes an RNA molecule which comprises tRNA-tracrRNA-tRNA-crRNA-tRNA-tracrRNA-tRNA-crRNA, wherein the crRNA and tracrRNA comprise the d9 and d11 (d9+d11) mutations, as described in Example 3.

SEQ ID NO: 44 is a DNA sequence of an expression cassette, driven by a prSoUbi promoter and terminated by an A. tumefaciens terminator, which encodes an RNA molecule which comprises tRNA-tracrRNA-tRNA-crRNA-tRNA-tracrRNA-tRNA-crRNA-tRNA, wherein the crRNA and tracrRNA comprise the d9+d11 mutations, as described in Example 3.

SEQ ID NO: 45 is the DNA sequence of the DEP1 protospacer target, which is the protospacer (and DNA-targeting segment) that is operably linked at the 5′ end of and is part of all the engineered crRNA molecules described in the Examples.

SEQ ID NOs: 46-48 are a PMI primer set and probe useful for the detection of a PMI gene.

SEQ ID NOs: 49-51 are a Cas9 primer set and probe useful for the detection of a Cas9 gene.

SEQ ID NOs: 52-54 are a OsDep1-2678 primer set and probe useful for the detection of targeted mutations in a OsDEP1 gene.

SEQ ID NOs: 55-76 are RNA sequences of the part of engineered crRNAs which does not include the DNA-targeting segment and its corresponding tracrRNA molecule, corresponding to DNA sequences of SEQ ID NOs: 3-24.

SEQ ID NOs: 77-84 are RNA sequences of the part of engineered crRNAs which does not include the DNA-targeting segment and its corresponding tracrRNA molecule, corresponding to DNA sequences of SEQ ID NOs: 28-35.

SEQ ID NO: 85 is the DNA sequence of a prSoUbi4 promoter, which is driven by a RNA polymerase II.

SEQ ID NO: 86 is the DNA sequence of a prOsU3 promoter, which is driven by a RNA polymerase III.

SEQ ID NOs: 87-95 are DNA sequences of expression cassettes similar to SEQ ID NOs: 25, 26, 37, 38, and 40-44, except 20 N's denote the coding sequence of the protospacer for the engineered crRNA. These N's can be any nucleotide, for example A, T, G, or C. These N's may also indicate no nucleotide, such that the protospacer may be less than 20 nucleotides in length.

SEQ ID NOs: 96-110 are RNA sequences of the duplex-forming segments of tracrRNA molecules of the invention.

SEQ ID NO: 111 is the DNA sequence of the Cas9 variant used for the examples.

SEQ ID NO: 112 is the DNA sequence which encodes for the pre-tRNA(gly) used as the tRNA sequences in the expression cassettes described in the examples.

### DETAILED DESCRIPTION OF THE INVENTION

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety.

The following definitions and methods are provided to better define the present invention and to guide those of ordinary skill in the art in the practice of the present invention. Unless otherwise noted, terms used herein are to be understood according to conventional usage by those of ordinary skill in the relevant art. Definitions of common terms in molecular biology may also be found in Rieger et al., Glossary of Genetics: Classical and Molecular, 5^{th} edition, Springer-Verlag: New York, 1994.

As used in the description of the embodiments of the invention and the appended claims, the singular forms “a,” “an,” and “the” are intended to include the plural forms as well, unless the context clearly indicates otherwise.

As used herein, “and/or” refers to and encompasses any and all possible combinations of one or more of the associated listed items.

The term “about,” as used herein when referring to a measurable value such as an amount of a compound, dose, time, temperature, and the like, is meant to encompass variations of 20%, 10%, 5%, 1%, 0.5%, or even 0.1% of the specified amount.

The terms “comprise,” “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the term “amplified” means the construction of multiple copies of a nucleic acid molecule or multiple copies complementary to the nucleic acid molecule using at least one of the nucleic acid molecules as a template. See, e.g., Diagnostic Molecular Microbiology: Principles and Applications, D. H. Persing et al., Ed., American Society for Microbiology, Washington, D.C. (1993). The product of amplification is termed an amplicon.

A “coding sequence” is a nucleic acid sequence that is transcribed into RNA such as mRNA, rRNA, tRNA, snRNA, sense RNA or antisense RNA. In some embodiments, the RNA is then translated in an organism to produce a protein.

“Expression cassette” as used herein means a nucleic acid molecule capable of directing expression of a particular nucleotide sequence in an appropriate host cell, comprising a promoter operably linked to the nucleotide sequence of interest, typically a coding region, which is operably linked to termination signals. It also typically comprises sequences required for proper translation of the nucleotide sequence. The coding region usually codes for a protein of interest but may also code for a functional RNA of interest, for example antisense RNA or a nontranslated RNA, such as a tRNA, in the sense or antisense direction. The expression cassette may also comprise sequences not necessary in the direct expression of a nucleotide sequence of interest but which are present due to convenient restriction sites for removal of the cassette from an expression vector. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the host cell or an ancestor of the host cell by a transformation process known in the art. The expression of the nucleotide sequence in the expression cassette may be under the control of a constitutive promoter or of an inducible promoter that initiates transcription only when the host cell is exposed to some particular external stimulus. In the case of a multicellular organism, such as a plant, the promoter can also be specific to a particular tissue, or organ, or stage of development. An expression cassette, or fragment thereof, can also be referred to as “inserted sequence” or “insertion sequence” when transformed into a plant.

A “gene” is a defined region that is located within a genome and that, besides the aforementioned coding nucleic acid sequence, comprises other, primarily regulatory, nucleic acid sequences responsible for the control of the expression, that is to say the transcription and translation, of the coding portion. Genes can include both coding and non-coding regions (e.g., introns, regulatory elements, promoters, enhancers, termination sequences and 5′ and 3′ untranslated regions). A gene typically expresses mRNA, functional RNA, or specific protein, including regulatory sequences. Genes may or may not be capable of being used to produce a functional protein. In some embodiments, a gene refers to only the coding region. The term “native gene” refers to a gene as found in nature. The term “chimeric gene” refers to any gene that contains 1) DNA sequences, including regulatory and coding sequences that are not found together in nature, or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature. A gene may be “isolated” by which is meant a nucleic acid molecule that is substantially or essentially free from components normally found in association with the nucleic acid molecule in its natural state. Such components include other cellular material, culture medium from recombinant production, and/or various chemicals used in chemically synthesizing the nucleic acid molecule.

By the term “express” or “expression” of a polynucleotide coding sequence, it is meant that the sequence is transcribed, and optionally translated.

A “gene of interest” or “nucleotide sequence of interest” refers to any gene which, when transferred to a plant, confers upon the plant a desired characteristic such as antibiotic resistance, virus resistance, insect resistance, disease resistance, or resistance to other pests, herbicide tolerance, improved nutritional value, improved performance in an industrial process or altered reproductive capability. The “gene of interest” may also be one that is transferred to plants for the production of commercially valuable enzymes or metabolites in the plant.

As used herein, “heterologous” refers to a nucleic acid molecule or nucleotide sequence not naturally associated with a host cell into which it is introduced, that either originates from another species or is from the same species or organism but is modified from either its original form or the form primarily expressed in the cell, including non-naturally occurring multiple copies of a naturally occurring nucleic acid sequence. Thus, a nucleotide sequence derived from an organism or species different from that of the cell into which the nucleotide sequence is introduced, is heterologous with respect to that cell and the cell's descendants. In addition, a heterologous nucleotide sequence includes a nucleotide sequence derived from and inserted into the same natural, original cell type, but which is present in a non-natural state, e.g., present in a different copy number, and/or under the control of different regulatory sequences than that found in the native state of the nucleic acid molecule. A nucleic acid sequence can also be heterologous to other nucleic acid sequences with which it may be associated, for example in a nucleic acid construct, such as e.g., an expression vector. As one non-limiting example, a promoter may be present in a nucleic acid construct in combination with one or more regulatory element and/or coding sequences that do not naturally occur in association with that particular promoter, i.e., they are heterologous to the promoter.

A “homologous” nucleic acid sequence is a nucleic acid sequence naturally associated with a host cell into which it is introduced. A homologous nucleic acid sequence can also be a nucleic acid sequence that is naturally associated with other nucleic acid sequences that may be present, e.g., in a nucleic acid construct. As one non-limiting example, a promoter may be present in a nucleic acid construct in combination with one or more regulatory elements and/or coding sequences that naturally occur in association with that particular promoter, i.e., they are homologous to the promoter.

“Operably-linked” refers to the association of nucleic acid sequences on a single nucleic acid sequence so that the function of one affects the function of the other. For example, a promoter is operably-linked with a coding sequence or functional RNA when it is capable of affecting the expression of that coding sequence or functional RNA (i.e., the coding sequence or functional RNA is under the transcriptional control of the promoter). Coding sequences in sense or antisense orientation can be operably-linked to regulatory sequences. Thus, regulatory or control sequences (e.g., promoters) operatively associated with a nucleotide sequence are capable of effecting expression of the nucleotide sequence. For example, a promoter operably linked to a nucleotide sequence encoding GFP would be capable of effecting the expression of that GFP nucleotide sequence. The control sequences need not be contiguous with the nucleotide sequence of interest, as long as they function to direct the expression thereof. Thus, for example, intervening untranslated, yet transcribed, sequences can be present between a promoter and a coding sequence, and the promoter sequence can still be considered “operably linked” to the coding sequence.

“Primers” as used herein are isolated nucleic acids that are annealed to a complementary target DNA strand by nucleic acid hybridization to form a hybrid between the primer and the target DNA strand, then extended along the target DNA strand by a polymerase, such as DNA polymerase. Primer pairs or sets can be used for amplification of a nucleic acid molecule, for example, by the polymerase chain reaction (PCR) or other nucleic-acid amplification methods.

A “probe” is an isolated nucleic acid molecule that is complementary to a portion of a target nucleic acid molecule and is typically used to detect and/or quantify the target nucleic acid molecule. Thus, in some embodiments, a probe can be an isolated nucleic acid molecule to which is attached a detectable moiety or reporter molecule, such as a radioactive isotope, ligand, chemiluminescence agent, fluorescence agent or enzyme. Probes according to the present invention can include not only deoxyribonucleic or ribonucleic acids but also polyamides and other probe materials that bind specifically to a target nucleic acid sequence and can be used to detect the presence of and/or quantify the amount of, that target nucleic acid sequence.

A TaqMan probe is designed such that it anneals within a DNA region amplified by a specific set of primers. As the Taq polymerase extends the primer and synthesizes the nascent strand from a single-strand template from 3′ to 5′ of the complementary strand, the 5′ to 3′ exonuclease of the polymerase extends the nascent strand through the probe and consequently degrades the probe that has annealed to the template. Degradation of the probe releases the fluorophore from it and breaks the close proximity to the quencher, thus relieving the quenching effect and allowing fluorescence of the fluorophore. Hence, fluorescence detected in the quantitative PCR thermal cycler is directly proportional to the fluorophore released and the amount of DNA template present in the PCR.

Primers and probes are generally between 5 and 100 nucleotides or more in length. In some embodiments, primers and probes can be at least 20 nucleotides or more in length, or at least 25 nucleotides or more, or at least 30 nucleotides or more in length. Such primers and probes hybridize specifically to a target sequence under optimum hybridization conditions as are known in the art. Primers and probes according to the present invention may have complete sequence complementarity with the target sequence, although probes differing from the target sequence and which retain the ability to hybridize to target sequences may be designed by conventional methods according to the invention.

Methods for preparing and using probes and primers are described, for example, in Molecular Cloning: A Laboratory Manual, 2nd ed., vol. 1-3, ed. Sambrook et al., Cold Spring Harbor Laboratory Press, Cold Spring Harbor, N.Y., 1989. PCR-primer pairs can be derived from a known sequence, for example, by using computer programs intended for that purpose.

The polymerase chain reaction (PCR) is a technique for “amplifying” a particular piece of DNA. In order to perform PCR, at least a portion of the nucleotide sequence of the DNA molecule to be replicated must be known. In general, primers or short oligonucleotides are used that are complementary (e.g., substantially complementary or fully complementary) to the nucleotide sequence at the 3′ end of each strand of the DNA to be amplified (known sequence). The DNA sample is heated to separate its strands and is mixed with the primers. The primers hybridize to their complementary sequences in the DNA sample. Synthesis begins (5′ to 3′ direction) using the original DNA strand as the template. The reaction mixture must contain all four deoxynucleotide triphosphates (dATP, dCTP, dGTP, dTTP) and a DNA polymerase. Polymerization continues until each newly-synthesized strand has proceeded far enough to contain the sequence recognized by the other primer. Once this occurs, two DNA molecules are created that are identical to the original molecule. These two molecules are heated to separate their strands and the process is repeated. Each cycle doubles the number of DNA molecules. Using automated equipment, each cycle of replication can be completed in less than 5 minutes. After 30 cycles, what began as a single molecule of DNA has been amplified into more than a billion copies (2³⁰=1.02×10⁹).

A quantitative polymerase chain reaction (qPCR), also referred to as real-time polymerase chain reaction, monitors the accumulation of a DNA product from a PCR reaction in real time. qPCR is a laboratory technique of molecular biology based on the polymerase chain reaction (PCR), which is used to amplify and simultaneously quantify a targeted DNA molecule. TaqMan is a system for qPCR. Even one copy of a specific sequence can be amplified and detected in PCR. The PCR reaction generates copies of a DNA template exponentially. This results in a quantitative relationship between the amount of starting target sequence and amount of PCR product accumulated at any particular cycle. Due to inhibitors of the polymerase reaction found with the template, reagent limitation or accumulation of pyrophosphate molecules, the PCR reaction eventually ceases to generate template at an exponential rate (i.e., the plateau phase), making the end point quantitation of PCR products unreliable. Therefore, duplicate reactions may generate variable amounts of PCR product. Only during the exponential phase of the PCR reaction is it possible to extrapolate back in order to determine the starting quantity of template sequence. The measurement of PCR products as they accumulate (i.e., real-time quantitative PCR) allows quantitation in the exponential phase of the reaction and therefore removes the variability associated with conventional PCR. In a real time PCR assay, a positive reaction is detected by accumulation of a fluorescent signal. For one or more specific sequences in a DNA sample, quantitative PCR enables both detection and quantification. The quantity can be either an absolute number of copies or a relative amount when normalized to DNA input or additional normalizing genes. Since the first documentation of real-time PCR, it has been used for an increasing and diverse number of applications including mRNA expression studies, DNA copy number measurements in genomic or viral DNAs, allelic discrimination assays, expression analysis of specific splice variants of genes and gene expression in paraffin-embedded tissues and laser captured micro-dissected cells.

As used herein, the term “cell” refers to any living cell. The cell may be a prokaryotic or eukaryotic cell. The cell may be isolated. The cell may or may not be capable of regenerating into an organism. The cell may be in the context of a tissue, callus, culture, organ, or part. In some embodiments, the cell may be a plant cell. A plant cell of the present invention can be in the form of an isolated single cell or can be a cultured cell or can be a part of a higher-organized unit such as, for example, a plant tissue or a plant organ. The plant cell may be derived from or part of an angiosperm or gymnosperm. In further embodiments, the plant cell may be a monocotyledonous plant cell, a dicotyledonous plant cell. The monocotyledonous plant cell may be, for example, a maize, rice, sorghum, sugarcane, barley, wheat, oat, turf grass, or ornamental grass cell. The dicotyledonous plant cell may be, for example, a tobacco, pepper, eggplant, sunflower, crucifer, flax, potato, cotton, soybean, sugar bee, or oilseed rape cell.

The term “plant part,” as used herein, includes but is not limited to embryos, pollen, ovules, seeds, leaves, stems, shoots, flowers, branches, fruit, kernels, ears, cobs, husks, stalks, roots, root tips, anthers. A plant part comprises plant cells. “Plant cells” includes plant cells that are intact in plants and/or parts of plants, plant protoplasts, plant tissues, plant cell tissue cultures, plant calli, plant clumps, and the like. As used herein, “shoot” refers to the above ground parts including the leaves and stems. Further, as used herein, “plant cell” refers to a structural and physiological unit of the plant, which comprises a cell wall and also may refer to a protoplast.

The term “introducing” or “introduce” in the context of a cell, prokaryotic cell, bacterial cell, eukaryotic cell, plant cell, plant and/or plant part means contacting a nucleic acid molecule with the cell, eukaryotic cell, plant, plant part, and/or plant cell in such a manner that the nucleic acid molecule gains access to the interior of the cell, eukaryotic cell, plant cell and/or a cell of the plant and/or plant part. Where more than one nucleic acid molecule is to be introduced these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into plant cells in a single transformation event, in separate transformation events, or, e.g., as part of a breeding protocol.

As used herein, the terms “transformed” and “transgenic” refer to any cell, prokaryotic cell, eukaryotic cell, plant, plant cell, callus, plant tissue, or plant part that contains all or part of at least one recombinant (e.g., heterologous) polynucleotide. In some embodiments, all or part of the recombinant polynucleotide is stably integrated into a chromosome or stable extra-chromosomal element, so that it is passed on to successive generations. For the purposes of the invention, the term “recombinant polynucleotide” refers to a polynucleotide that has been altered, rearranged, or modified by genetic engineering. Examples include any cloned polynucleotide, or polynucleotides, that are linked or joined to heterologous sequences. The term “recombinant” does not refer to alterations of polynucleotides that result from naturally occurring events, such as spontaneous mutations, or from non-spontaneous mutagenesis followed by selective breeding.

The term “transformation” as used herein refers to the introduction of a heterologous nucleic acid into a cell. Transformation of a cell may be stable or transient. Thus, a transgenic cell, plant cell, plant and/or plant part of the invention can be stably transformed or transiently transformed. Transformation can refer to the transfer of a nucleic acid molecule into the genome of a host cell, resulting in genetically stable inheritance. In some embodiments, the introduction into a plant, plant part and/or plant cell is via bacterial-mediated transformation, particle bombardment transformation, calcium-phosphate-mediated transformation, cyclodextrin-mediated transformation, electroporation, liposome-mediated transformation, nanoparticle-mediated transformation, polymer-mediated transformation, virus-mediated nucleic acid delivery, whisker-mediated nucleic acid delivery, microinjection, sonication, infiltration, polyethylene glycol-mediated transformation, protoplast transformation, or any other electrical, chemical, physical and/or biological mechanism that results in the introduction of nucleic acid into the plant, plant part and/or cell thereof, or any combination thereof.

Procedures for transforming plants are well known and routine in the art and are described throughout the literature. Non-limiting examples of methods for transformation of plants include transformation via bacterial-mediated nucleic acid delivery (e.g., via bacteria from the genus Agrobacterium), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Mild et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell Mol Biol Lett 7:849-858 (2002)).

Agrobacterium-mediated transformation is a commonly used method for transforming plants because of its high efficiency of transformation and because of its broad utility with many different species. Agrobacterium-mediated transformation typically involves transfer of the binary vector carrying the foreign DNA of interest to an appropriate Agrobacterium strain that may depend on the complement of vir genes carried by the host Agrobacterium strain either on a co-resident Ti plasmid or chromosomally (Uknes et al. 1993, Plant Cell 5:159-169). The transfer of the recombinant binary vector to Agrobacterium can be accomplished by a tri-parental mating procedure using Escherichia coli carrying the recombinant binary vector, a helper E. coli strain that carries a plasmid that is able to mobilize the recombinant binary vector to the target Agrobacterium strain. Alternatively, the recombinant binary vector can be transferred to Agrobacterium by nucleic acid transformation (Höfgen and Willmitzer 1988, Nucleic Acids Res 16:9877).

Transformation of a plant by recombinant Agrobacterium usually involves co-cultivation of the Agrobacterium with explants from the plant and follows methods well known in the art. Transformed tissue is typically regenerated on selection medium carrying an antibiotic or herbicide resistance marker between the binary plasmid T-DNA borders.

Another method for transforming plants, plant parts and plant cells involves propelling inert or biologically active particles at plant tissues and cells. See, e.g., U.S. Pat. Nos. 4,945,050, 5,036,006 and 5,100,792. Generally, this method involves propelling inert or biologically active particles at the plant cells under conditions effective to penetrate the outer surface of the cell and afford incorporation within the interior thereof. When inert particles are utilized, the vector can be introduced into the cell by coating the particles with the vector containing the nucleic acid of interest. Alternatively, a cell or cells can be surrounded by the vector so that the vector is carried into the cell by the wake of the particle. Biologically active particles (e.g., dried yeast cells, dried bacteria or a bacteriophage, each containing one or more nucleic acids sought to be introduced) also can be propelled into plant tissue.

“Transient transformation” in the context of a polynucleotide means that a polynucleotide is introduced into the cell and does not integrate into the genome of the cell. Transient transformation may be detected by, for example, an enzyme-linked immunosorbent assay (ELISA) or Western blot, which can detect the presence of a peptide or polypeptide encoded by one or more nucleic acid molecules introduced into an organism.

As used herein, “stably introducing,” “stably introduced,” “stable transformation” or “stably transformed” in the context of a polynucleotide introduced into a cell, means that the introduced polynucleotide is stably integrated into the genome of the cell, and thus the cell is stably transformed with the polynucleotide. As such, the integrated polynucleotide is capable of being inherited by the progeny thereof, more particularly, by the progeny of multiple successive generations. “Genome” as used herein includes the nuclear and/or plastid genome, and therefore includes integration of a polynucleotide into, for example, the chloroplast genome. Stable transformation as used herein can also refer to a polynucleotide that is maintained extrachromasomally, for example, as a minichromosome.

Stable transformation of a cell can be detected by, for example, a Southern blot hybridization assay of genomic DNA of the cell with nucleic acid sequences which specifically hybridize with a nucleotide sequence of a nucleic acid molecule introduced into an organism (e.g., a plant). Stable transformation of a cell can be detected by, for example, a Northern blot hybridization assay of RNA of the cell with nucleic acid sequences which specifically hybridize with a nucleotide sequence of a nucleic acid molecule introduced into a plant or other organism. Stable transformation of a cell can also be detected by, e.g., a polymerase chain reaction (PCR) or other amplification reaction as are well known in the art, employing specific primer sequences that hybridize with target sequence(s) of a nucleic acid molecule, resulting in amplification of the target sequence(s), which can be detected according to standard methods. Transformation can also be detected by direct sequencing and/or hybridization protocols well known in the art.

Thus, in particular embodiments of the present invention, a plant cell can be transformed by any method known in the art and as described herein and intact plants can be regenerated from these transformed cells using any of a variety of known techniques. Plant regeneration from plant cells, plant tissue culture and/or cultured protoplasts is described, for example, in Evans et al. (Handbook of Plant Cell Cultures, Vol. 1, MacMilan Publishing Co. New York (1983)); and Vasil I. R. (ed.) (Cell Culture and Somatic Cell Genetics of Plants, Acad. Press, Orlando, Vol. I (1984), and Vol. II (1986)). Methods of selecting for transformed transgenic plants, plant cells and/or plant tissue culture are routine in the art and can be employed in the methods of the invention provided herein.

The “transformation and regeneration process” refers to the process of stably introducing a transgene into a plant cell and regenerating a plant from the transgenic plant cell. As used herein, transformation and regeneration includes the selection process, whereby a transgene comprises a selectable marker and the transformed cell has incorporated and expressed the transgene, such that the transformed cell will survive and developmentally flourish in the presence of the selection agent. “Regeneration” refers to growing a whole plant from a plant cell, a group of plant cells, or a plant piece such as from a protoplast, callus, or tissue part.

The terms “nucleotide sequence” “nucleic acid,” “nucleic acid sequence,” “nucleic acid molecule,” “oligonucleotide” and “polynucleotide” are used interchangeably herein to refer to a heteropolymer of nucleotides and encompass both RNA and DNA, including cDNA, genomic DNA, mRNA, synthetic (e.g., chemically synthesized) DNA or RNA and chimeras of RNA and DNA. The term nucleic acid molecule refers to a chain of nucleotides without regard to length of the chain. The nucleotides contain a sugar, phosphate and a base which is either a purine or pyrimidine. A nucleic acid molecule can be double-stranded or single-stranded. Where single-stranded, the nucleic acid molecule can be a sense strand or an antisense strand. A nucleic acid molecule can be synthesized using oligonucleotide analogs or derivatives (e.g., inosine or phosphorothioate nucleotides). Such oligonucleotides can be used, for example, to prepare nucleic acid molecules that have altered base-pairing abilities or increased resistance to nucleases. Nucleic acid sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25.

A “nucleic acid fragment” is a fraction of a given nucleic acid molecule. An “RNA fragment” is a fraction of a given RNA molecule. A “DNA fragment” is a fraction of a given DNA molecule. A “nucleic acid segment” is a fraction of a given nucleic acid molecule and is not isolated from the molecule. An “RNA segment” is a fraction of a given RNA molecule and is not isolated from the molecule. A “DNA segment” is a fraction of a given DNA molecule and is not isolated from the molecule. Segments of polynucleotides can be any length, for example, at least 5, 10, 15, 20, 25, 30, 40, 50, 75, 100, 150, 200, 300 or 500 or more nucleotides in length. A segment or portion of a guide sequence can be about 50%, 40%, 30%, 20%, 10% of the guide sequence, e.g., one-third of the guide sequence or shorter, e.g., 7, 6, 5, 4, 3, or 2 nucleotides in length.

The term “derived from” in the context of a molecule refers to a molecule isolated or made using a parent molecule or information from that parent molecule. For example, a Cas9 single mutant nickase and a Cas9 double mutant null-nuclease are each derived from a wild-type Cas9 protein.

In higher plants, deoxyribonucleic acid (DNA) is the genetic material while ribonucleic acid (RNA) is involved in the transfer of information contained within DNA into proteins. A “genome” is the entire body of genetic material contained in each cell of an organism. Unless otherwise indicated, a particular nucleic acid sequence of this invention also implicitly encompasses conservatively modified variants thereof (e.g., degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid molecule is used interchangeably with gene, cDNA, and mRNA encoded by a gene.

As used herein “sequence identity” refers to the extent to which two optimally aligned polynucleotide or peptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence.

As used herein, the phrase “substantially identical,” in the context of two nucleic acid molecules, nucleotide sequences or protein sequences, refers to two or more sequences or subsequences that have at least about 70%, least about 75%, at least about 80%, least about 85%, at least about 90%, at least about 95%, at least about 96%, at least about 97%, at least about 98%, or at least about 99% nucleotide or amino acid residue identity, when compared and aligned for maximum correspondence, as measured using one of the following sequence comparison algorithms or by visual inspection. In some embodiments of the invention, the substantial identity exists over a region of the sequences that is at least about 15 residues to about 150 residues in length. Thus, in some embodiments of this invention, the substantial identity exists over a region of the sequences that is at least about 10, about 20, about 30, about 40, 50, about 60, about 70, about 80, about 90, about 100, about 110, about 120, about 130, about 140, about 150, or more residues in length. In some particular embodiments, the sequences are substantially identical over at least about 80 residues. In a further embodiment, the sequences are substantially identical over the entire length of the coding regions, for example over the entire length of a tracrRNA molecule. Furthermore, in representative embodiments, substantially identical nucleotide or protein sequences perform substantially the same function (e.g., guiding to a particular genomic target, endonuclease cleavage of a particular genomic target site).

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, Calif.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleotide sequence to the reference nucleotide sequence is less than about 0.1 to less than about 0.001. Thus, in some embodiments of the invention, the smallest sum probability in a comparison of the test nucleotide sequence to the reference nucleotide sequence is less than about 0.001.

Two nucleotide sequences can also be considered to be substantially identical when the two sequences hybridize to each other under stringent conditions. In some representative embodiments, two nucleotide sequences considered to be substantially identical hybridize to each other under highly stringent conditions.

“Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.15M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

The following are examples of sets of hybridization/wash conditions that may be used to clone homologous nucleotide sequences that are substantially identical to reference nucleotide sequences of the present invention. In one embodiment, a reference nucleotide sequence hybridizes to the “test” nucleotide sequence in 7% sodium dodecyl sulfate (SDS), 0.5 M NaPO₄, 1 mM EDTA at 50° C. with washing in 2×SSC, 0.1% SDS at 50° C. In another embodiment, the reference nucleotide sequence hybridizes to the “test” nucleotide sequence in 7% sodium dodecyl sulfate (SDS), 0.5 M NaPO₄, 1 mM EDTA at 50° C. with washing in 1×SSC, 0.1% SDS at 50° C. or in 7% sodium dodecyl sulfate (SDS), 0.5 M NaPO₄, 1 mM EDTA at 50° C. with washing in 0.5×SSC, 0.1% SDS at 50° C. In still further embodiments, the reference nucleotide sequence hybridizes to the “test” nucleotide sequence in 7% sodium dodecyl sulfate (SDS), 0.5 M NaPO₄, 1 mM EDTA at 50° C. with washing in 0.1×SSC, 0.1% SDS at 50° C., or in 7% sodium dodecyl sulfate (SDS), 0.5 M NaPO₄, 1 mM EDTA at 50° C. with washing in 0.1×SSC, 0.1% SDS at 65° C.

An “isolated” nucleic acid molecule or nucleotide sequence or an “isolated” polypeptide is a nucleic acid molecule, nucleotide sequence or polypeptide that, by the hand of man, exists apart from its native environment and/or has a function that is different, modified, modulated and/or altered as compared to its function in its native environment and is therefore not a product of nature. An isolated nucleic acid molecule or isolated polypeptide may exist in a purified form or may exist in a non-native environment such as, for example, a recombinant host cell. Thus, for example, with respect to polynucleotides, the term isolated means that it is separated from the chromosome and/or cell in which it naturally occurs. A polynucleotide is also isolated if it is separated from the chromosome and/or cell in which it naturally occurs and is then inserted into a genetic context, a chromosome, a chromosome location, and/or a cell in which it does not naturally occur. The recombinant nucleic acid molecules and nucleotide sequences of the invention can be considered to be “isolated” as defined above.

“Wild-type” nucleotide sequence or amino acid sequence refers to a naturally occurring (“native”) or endogenous nucleotide sequence or amino acid sequence. Thus, for example, a “wild-type mRNA” is an mRNA that is naturally occurring in or endogenous to the organism. A “homologous” nucleotide sequence is a nucleotide sequence naturally associated with a host cell into which it is introduced.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

“Promoter” refers to a nucleotide sequence, usually upstream (5′) to its coding sequence, which controls the expression of the coding sequence by providing the recognition for RNA polymerase and other factors required for proper transcription. “Promoter regulatory sequences” consist of proximal and more distal upstream elements. Promoter regulatory sequences influence the transcription, RNA processing or stability, or translation of the associated coding sequence. Regulatory sequences include enhancers, promoters, untranslated leader sequences, introns, and polyadenylation signal sequences. They include natural and synthetic sequences as well as sequences that may be a combination of synthetic and natural sequences. An “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. The meaning of the term “promoter” includes “promoter regulatory sequences.”

“Primary transformant” and “T0 generation” refer to transgenic plants that are of the same genetic generation as the tissue that was initially transformed (i.e., not having gone through meiosis and fertilization since transformation). “Secondary transformants” and the “T1, T2, T3, etc. generations” refer to transgenic plants derived from primary transformants through one or more meiotic and fertilization cycles. They may be derived by self-fertilization of primary or secondary transformants or crosses of primary or secondary transformants with other transformed or untransformed plants.

A “transgene” refers to a nucleic acid molecule that has been introduced into the genome by transformation and is stably maintained. A transgene may comprise at least one expression cassette, typically comprises at least two expression cassettes, and may comprise ten or more expression cassettes. Transgenes may include, for example, genes that are either heterologous or homologous to the genes of a particular plant to be transformed. Additionally, transgenes may comprise native genes inserted into a non-native organism, or chimeric genes. The term “endogenous gene” refers to a native gene in its natural location in the genome of an organism. A “foreign” gene refers to a gene not normally found in the host organism but one that is introduced into the organism by gene transfer.

“Intron” refers to an intervening section of DNA which occurs almost exclusively within a eukaryotic gene, but which is not translated to amino acid sequences in the gene product. The introns are removed from the pre-mature mRNA through a process called splicing, which leaves the exons untouched, to form an mRNA. For purposes of the present invention, the definition of the term “intron” includes modifications to the nucleotide sequence of an intron derived from a target gene, provided the modified intron does not significantly reduce the activity of its associated 5′ regulatory sequence.

“Exon” refers to a section of DNA which carries the coding sequence for a protein or part of it. Exons are separated by intervening, non-coding sequences (introns). For purposes of the present invention, the definition of the term “exon” includes modifications to the nucleotide sequence of an exon derived from a target gene, provided the modified exon does not significantly reduce the activity of its associated 5′ regulatory sequence.

As used herein, a “target site,” “target sequence”, or “target protospacer DNA” are used interchangeably herein to refer to a nucleic acid sequence present in a target DNA to which a DNA targeting segment of a subject DNA-targeting RNA will bind, provided sufficient conditions for binding exist. Suitable DNA/RNA binding conditions include physiological conditions normally present in a cell. Other suitable DNA/RNA binding conditions (e.g., conditions in a cell-free system) are known in the art; see, e.g., Sambrook, supra. The strand of the target DNA that is complementary to and hybridizes with the DNA-targeting RNA is referred to as the “complementary strand” and the strand of the target DNA that is complementary to the “complementary strand” (and is therefore not complementary to the DNA-targeting RNA) is referred to as the “non-complementary strand.”

As used herein, the terms “proximal” or “proximal to” with regard to one or more nucleotide sequences of this invention means immediately next to (e.g., with no intervening sequence) or separated by from about 1 base to about 10,000 bases (e.g., 1, 2, 3, 4, 5, 6, 7, 8, 9, 10, 15, 20, 30, 40, 50, 100, 200, 500, 1000, 2000, 3000, 4000, 5000, 6000, 7000, 8000, 9000, or 10,000 bases), including any values included within this range but not explicitly recited herein.

The term “cleavage” or “cleaving” refers to breaking of the covalent phosphodiester linkage in the ribosylphosphodiester backbone of a polynucleotide. The terms “cleavage” or “cleaving” encompass both single-stranded breaks and double-stranded breaks. Double-stranded cleavage can occur as a result of two distinct single-stranded cleavage events. Cleavage can result in the production of either blunt ends or staggered ends. A “nuclease cleavage site” or “genomic nuclease cleavage site” is a region of nucleotides that comprise a nuclease cleavage sequence that is recognized by a specific nuclease, which acts to cleave the nucleotide sequence of the genomic DNA in one or both strands. Such cleavage by the nuclease enzyme initiates DNA repair mechanisms within the cell, which establishes an environment for homologous recombination or non-homologous end-joining to occur.

The terms “CRISPR-associated protein”, “Cas protein”, “CRISPR-associated nuclease” or “Cas nuclease” refer to a wild type Cas protein, a fragment thereof, or a mutant or variant thereof. The term “Cas mutant” or “Cas variant” refers to a protein or polypeptide derivative of a wild type Cas protein, e.g., a protein having one or more point mutations, insertions, deletions, truncations, a fusion protein, or a combination thereof. In certain embodiments, the Cas mutant or Cas variant substantially retains the nuclease activity of the Cas protein, such as a Cas9 variant described herein which is operably linked to a nuclear localization signal (NLS) derived from a plant. In certain embodiments, the Cas nuclease is mutated such that one or both nuclease domains are inactive, such as, for example, a catalytically dead Cas9 referred to as dCas9, which is still able to target to a specific genomic location but has no endonuclease activity (Qi et al., 2013, Cell, 152: 1173-1183, hereby incorporated within). In some embodiments, the Cas nuclease is mutated so that it lacks some or all of the nuclease activity of its wild-type counterpart. The Cas protein may be Cas9, Cpf1 (Zetsche et al., 2015, Cell, 163: 759-771, hereby incorporated within) or another CRISPR-associated nuclease.

A “donor molecule”, “donor polynucleotide”, or “donor sequence” is a nucleotide polymer or oligomer intended for insertion at a target polynucleotide, typically a target genomic site. The donor sequence may be one or more transgenes, expression cassettes, or nucleotide sequences of interest. A donor molecule may be a donor DNA molecule, either single stranded, partially double-stranded, or double-stranded. The donor polynucleotide may be a natural or a modified polynucleotide, a RNA-DNA chimera, or a DNA fragment, either single- or at least partially double-stranded, or a fully double-stranded DNA molecule, or a PGR amplified ssDNA or at least partially dsDNA fragment. In some embodiments, the donor DNA molecule is part of a circularized DNA molecule. A fully double-stranded donor DNA is advantageous since it might provide an increased stability, since dsDNA fragments are generally more resistant than ssDNA to nuclease degradation. The donor molecule may comprise at least 10 contiguous nucleotides, wherein the nucleic acid molecule is at least 70% identical to a genomic nucleotide sequence, such that these contiguous nucleotides are sufficient for homologous recombination of the donor DNA molecule into the genome of the cell at the targeted genomic DNA sequence following cleavage by the site-directed modifying polypeptide (in this case, a site-directed nuclease). In some embodiments, the donor DNA molecule can comprise at least about 10, 20, 30, 50, 70, 80, 100, 150, 200, 250, 300, 250, 400, 450, 500, 600, 700, 800, 900, 1000, 1500, 2000, 2500, 3000, 3500, 4000, 4500, 5000, 7500, 10000, 15,000 or 20,000 nucleotides, including any value within this range not explicitly recited herein, wherein the donor DNA molecule is at least 70%, 75%, 80%, 85%, 90%, 91%, 92%, 93%, 94%, 95%, 96%, 97%, 98%, 99%, or 100% identical to a genomic nucleic acid sequence. In some embodiments, the donor DNA molecule may be substantially complementary to a genomic nucleic acid sequence. In some embodiments, the donor DNA molecule comprises heterologous nucleic acid sequence. In some embodiments, the donor DNA molecule comprises at least one expression cassette. In some embodiments, the donor DNA molecule may comprise a transgene, which comprises at least one expression cassette. In some embodiments, the donor DNA molecule comprises an allelic modification of a gene which is native to the target genome. The allelic modification may comprise at least one nucleotide insertion, at least one nucleotide deletion, and/or at least one nucleotide substitution. In some embodiments, the allelic modification may comprise an INDEL. In some embodiments, the donor DNA molecule comprises homologous arms to the target genomic site. In some embodiments, the donor DNA molecule comprises at least 100 contiguous nucleotides at least 90% identical to a genomic nucleic acid sequence, and optionally may further comprise a heterologous nucleic acid sequence such as a transgene.

As used herein, “site-directed modifying polypeptide” or “RNA binding site-directed polypeptide” or “RNA-binding site-directed modifying polypeptide” refers to a polypeptide that binds RNA and is targeted to a specific DNA sequence. An example of a site-directed modifying polypeptide is a CRISPR-associated nuclease, for example a Cas9 or a variant thereof. A site-directed modifying polypeptide as described herein is targeted to a specific DNA sequence by the RNA molecule(s) to which it is bound. The RNA molecule, or RNA duplex if it is two RNA molecules, comprises a sequence that is complementary to a target sequence within the target DNA, thus targeting the bound polypeptide to a specific location within the target DNA (the target sequence).

The RNA duplex that binds to the site-directed modifying polypeptide and targets the polypeptide to a specific location within the target DNA is referred to herein as the “DNA-targeting RNA duplex.” A DNA-targeting RNA duplex of the invention comprises two molecules, which together provide a “DNA-targeting segment” and a “protein-binding segment.” The two molecules are known in the art as the crRNA (“CRISPR RNA”) and the tracrRNA (“trans-acting CRISPR RNA) (Jinek et al., 2012). A crRNA molecule comprises both the DNA-targeting segment (single stranded) of the DNA-targeting RNA duplex and a stretch (“duplex-forming segment”) of nucleotides that forms one half of the RNA duplex of the protein-binding segment of the DNA-targeting RNA duplex. A corresponding tracrRNA molecule comprises a stretch of nucleotides (duplex-forming segment) that forms the other half of the RNA duplex of the protein-binding segment of the DNA-targeting RNA duplex. In other words, a stretch of nucleotides of a crRNA molecule are complementary to and hybridize with a stretch of nucleotides of a tracrRNA molecule to form the RNA duplex of the protein-binding domain of the DNA-targeting RNA. As such, each crRNA molecule can be said to have a corresponding tracrRNA molecule. The crRNA molecule additionally provides the single stranded DNA-targeting segment. Thus, a crRNA molecule and a tracrRNA molecule, as a corresponding pair, hybridize to form a DNA-targeting RNA duplex. A DNA-targeting RNA duplex can comprise any corresponding crRNA and tracrRNA pair.

The term “duplex-forming segment” is used herein to mean the stretch of nucleotides of a crRNA molecule or of a tracrRNA molecule that contributes to the formation of the RNA duplex by hybridizing to a stretch of nucleotides of a corresponding crRNA or tracrRNA molecule. In other words, a crRNA comprises a duplex-forming segment that is complementary to the duplex-forming segment of the corresponding tracrRNA. As such, a tracrRNA comprises a duplex-forming segment while a crRNA comprises both a duplex-forming segment and the DNA-targeting segment of the DNA-targeting RNA duplex.

By “segment” it is meant a segment/section/region of a molecule, e.g., a contiguous stretch of nucleotides in an RNA. A segment can also mean a region/section of a complex such that a segment may comprise regions of more than one molecule. For example, the protein-binding segment of a DNA-targeting RNA duplex comprises two separate RNA molecules that are hybridized along a region of complementarity. As an illustrative, non-limiting example, a protein-binding segment of a DNA-targeting RNA duplex can comprise (i) base pairs 40-75 of a first RNA molecule that is 100 base pairs in length; and (ii) base pairs 10-25 of a second RNA molecule that is 50 base pairs in length. The definition of “segment,” unless otherwise specifically defined in a particular context, is not limited to a specific number of total base pairs, is not limited to any particular number of base pairs from a given RNA molecule, is not limited to a particular number of separate molecules within a complex, and may include regions of RNA molecules that are of any total length and may or may not include regions with complementarity to other molecules. The DNA-targeting segment (or “DNA-targeting sequence”) of a DNA-targeting RNA duplex of the invention comprises a nucleotide sequence that is complementary to a specific sequence within a target DNA (the complementary strand of the target DNA). The protein-binding segment (or “protein-binding sequence”) interacts with a site-directed modifying polypeptide.

When the site-directed modifying polypeptide is a Cas9 or Cas9 variant polypeptide, site-specific cleavage of the target DNA occurs at locations determined by both (i) basepairing complementarity between the DNA-targeting segment of the crRNA and the target DNA; and (ii) a short motif (referred to as the protospacer adjacent motif (PAM)) in the target DNA. The protein-binding segment of the DNA-targeting RNA duplex comprises two complementary stretches of nucleotides that hybridize to one another to form a double stranded RNA duplex (dsRNA duplex). An exemplary DNA-targeting RNA duplex comprises a crRNA molecule and a corresponding tracrRNA molecule. A DNA-targeting RNA molecule may also comprise a single molecule, for example the so-called single guide RNA, which is capable of forming a secondary structure that includes a protein-binding segment formed by hybridization of opposite ends of the single guide RNA molecule (Jinek et al., 2012).

The present disclosure provides a DNA-targeting RNA duplex that directs the activities of an associated a site-directed modifying polypeptide to a specific target sequence within a target DNA. The DNA-targeting RNA duplex of the invention comprises two RNA molecules, namely a crRNA and a tracrRNA molecule. The crRNA comprises DNA-targeting segment which is a nucleic acid sequence that is complementary to a sequence in a target DNA. This DNA-targeting segment is also referred to as the “protospacer”. In other words, the DNA-targeting segment of a crRNA molecule of the invention interacts with a target DNA in a sequence-specific manner via hybridization (i.e., base pairing). As such, the nucleotide sequence of the DNA-targeting segment of the crRNA molecule may vary and determines the location within the target DNA that the DNA-targeting RNA duplex and the target DNA will interact.

The DNA targeting segment of a crRNA molecule of the invention can be modified (e.g., by genetic engineering) to hybridize to any desired sequence within a target DNA. The DNA-targeting segment of a crRNA molecule of the invention can have a length from about 12 nucleotides to about 100 nucleotides. For example, the DNA-targeting segment of a crRNA of the invention can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, or from about 12 nt to about 19 nt. For example, the DNA-targeting segment of a crRNA of the invention can have a length of from about 17 nt to about 27 nts. For example, the DNA-targeting segment of a crRNA of the invention can have a length of from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 19 nt to about 70 nt, from about 19 nt to about 80 nt, from about 19 nt to about 90 nt, from about 19 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt. The nucleotide sequence of the DNA-targeting segment of a crRNA of the invention can have a length at least about 12 nt. In some embodiments, the DNA-targeting segment of a crRNA of the invention is 20 nucleotides in length. In some embodiments, the DNA-targeting segment of a crRNA of the invention is 19 nucleotides in length.

In SEQ ID NOs: 87-95, which comprise DNA sequences encoding expression cassettes for the expression of at least one crRNA and at least one tracrRNA, the DNA-targeting segment of the crRNA is denoted with twenty “N”s. These twenty N's are understood to represent the DNA-targeting segment of a crRNA of the invention, and can be modified to hybridize to any desired sequence within a target DNA. These twenty N's are also understood to represent a length suitable for a DNA-targeting segment, as described above and as well-known to a person of skill in the art, wherein the length is at least about 12 nt, at least about 15 nt, at least about 18 nt, at least about 19 nt, at least about 20 nt, at least about 25 nt, at least about 30 nt, at least about 35 nt, at least about 40 nt, at least about 50 nt, at least about 60 nt, at least about 70 nt, at least about 80 nt, at least about 90 nt, or at least about 100 nt.

The percent complementarity between the DNA-targeting segment and the target sequence of the target DNA can be at least 60% (e.g., at least 65%, at least 70%, at least 75%, at least 80%, at least 85%, at least 90%, at least 95%, at least 97%, at least 98%, at least 99%, or 100%). In some cases, the percent complementarity between the DNA-targeting segment of a crRNA of the invention and the target sequence of the target DNA is 100% over the seven contiguous 5′-most nucleotides of the target sequence of the complementary strand of the target DNA. In some cases, the percent complementarity between the DNA-targeting sequence of the DNA-targeting segment and the target sequence of the target DNA is at least 60% over about 20 contiguous nucleotides. In some cases, the percent complementarity between the DNA-targeting segment of the crRNA and the target sequence of the target DNA is 100% over the fourteen contiguous 5′-most nucleotides of the target sequence of the complementary strand of the target DNA and as low as 0% over the remainder. In such a case, the DNA-targeting sequence can be considered to be 14 nucleotides in length. In some cases, the percent complementarity between the DNA-targeting segment of the crRNA and the target sequence of the target DNA is 100% over the seven contiguous 5′-most nucleotides of the target sequence of the complementary strand of the target DNA and as low as 0% over the remainder. In such a case, the DNA-targeting sequence can be considered to be 7 nucleotides in length.

The protein-binding segment of a DNA-targeting RNA duplex of the invention interacts with a site-directed modifying polypeptide. A DNA-targeting RNA duplex guides the bound polypeptide to a specific nucleotide sequence within a target DNA via the above mentioned DNA-targeting segment of the crRNA molecule. The protein-binding segment of a DNA-targeting RNA duplex of the invention comprises two stretches of nucleotides that are complementary to one another. One of these stretches is on the crRNA molecule, and the other is on the tracrRNA molecule. Each of these stretches of nucleotides are complementary to one another such that the complementary nucleotides of the two RNA molecules hybridize to form the double stranded RNA duplex of the protein-binding segment. The protein-binding segment comprises the duplex-forming segments of the crRNA molecule and tracrRNA molecule, where they hybridize to form a double-stranded RNA segment, which is then recognized by the site-directed modifying polypeptide for binding. The duplex-forming segment may include secondary RNA structures, represented in the primary RNA sequences as mismatches between the crRNA and tracrRNA molecules and/or 5′ overhangs (for the tracrRNA molecule) and/or 3′ overhangs (for the crRNA molecule).

The crRNA is comprised of the duplex-forming segment and the protospacer. In some embodiment, the duplex-forming segment of a crRNA of the invention is at least 80% identical, at least 85% identical, at least 90% identical, at least 91%, at least 92%, at least 93%, at least 94%, at least 95%, at least 96%, at least 97%, at least 98%, at least 99% or 100% identical to SEQ ID NOs: 55, 57, 59, 61, 63, 65, 67, 69, 71, 73, 75, 77, 79, 81, 83, or a complement thereof, over a stretch of at least 8, at least 10, at least 12, at least 14, at least 16, at least 18, at least 20, at least 22, or 22 contiguous nucleotides.

The corresponding duplex-forming segment of the tracrRNA is at the 5′ end of the tracrRNA molecule. In some embodiments, the duplex-forming segment of the tracrRNA molecule is at least 80% identical, at least 85% identical, at least 90% identical, at least 91%, at least 92%, at least 93%, at least 94%, at least 95%, at least 96%, at least 97%, at least 98%, at least 99% or 100% identical to SEQ ID NOs: 96-110, or a complement thereof, over a stretch of at least 8, at least 10, at least 12, at least 14, at least 16, at least 18, at least 20, at least 22, or 22 contiguous nucleotides. For example, the duplex forming segment of the tracrRNA (or the DNA encoding the duplex-forming segment of the tracrRNA) is at least about 65% identical, at least about 70% identical, at least about 75% identical, at least about 80% identical, at least about 85% identical, at least about 90% identical, at least about 95% identical, at least about 98% identical, at least about 99% identical or 100% identical to one of the duplex-forming segments of tracrRNA sequences set forth in SEQ ID NOs: 96-110, or a complement thereof, over a stretch of at least 8, at least 10, at least 12, at least 14, at least 16, at least 18, at least 19, at least 20, at least 21, or at least 22 contiguous nucleotides.

The two duplex-forming segments of the crRNA and tracrRNA molecules hybridize and form the protein-binding segment. In some embodiments, the duplex-forming segment of the crRNA molecule is at least 60% identical, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical, to a corresponding tracrRNA molecule of the invention, over a stretch of at least 8, at least 10, at least 12, at least 14, at least 16, at least 18, at least 19, at least 20, at least 21, or at least 22 contiguous nucleotides.

In some embodiments, the duplex-forming segment of the crRNA and the corresponding duplex-forming segment of the tracrRNA are SEQ ID NOs: 55 and 96, respectively. In some embodiments, the duplex-forming segment of the crRNA and the corresponding duplex-forming segment of the tracrRNA are SEQ ID NOs: 57 and 97, respectively. In some embodiments, the duplex-forming segment of the crRNA and the corresponding duplex-forming segment of the tracrRNA are SEQ ID NOs: 59 and 98, respectively. In some embodiments, the duplex-forming segment of the crRNA and the corresponding duplex-forming segment of the tracrRNA are SEQ ID NOs: 61 and 99, respectively. In some embodiments, the duplex-forming segment of the crRNA and the corresponding duplex-forming segment of the tracrRNA are SEQ ID NOs: 63 and 100, respectively. In some embodiments, the duplex-forming segment of the crRNA and the corresponding duplex-forming segment of the tracrRNA are SEQ ID NOs: 65 and 101, respectively. In some embodiments, the duplex-forming segment of the crRNA and the corresponding duplex-forming segment of the tracrRNA are SEQ ID NOs: 67 and 102, respectively. In some embodiments, the duplex-forming segment of the crRNA and the corresponding duplex-forming segment of the tracrRNA are SEQ ID NOs: 69 and 103, respectively. In some embodiments, the duplex-forming segment of the crRNA and the corresponding duplex-forming segment of the tracrRNA are SEQ ID NOs: 71 and 104, respectively. In some embodiments, the duplex-forming segment of the crRNA and the corresponding duplex-forming segment of the tracrRNA are SEQ ID NOs: 73 and 105, respectively. In some embodiments, the duplex-forming segment of the crRNA and the corresponding duplex-forming segment of the tracrRNA are SEQ ID NOs: 75 and 106, respectively. In some embodiments, the duplex-forming segment of the crRNA and the corresponding duplex-forming segment of the tracrRNA are SEQ ID NOs: 77 and 107, respectively. In some embodiments, the duplex-forming segment of the crRNA and the corresponding duplex-forming segment of the tracrRNA are SEQ ID NOs: 79 and 108, respectively. In some embodiments, the duplex-forming segment of the crRNA and the corresponding duplex-forming segment of the tracrRNA are SEQ ID NOs: 81 and 109, respectively. In some embodiments, the duplex-forming segment of the crRNA and the corresponding duplex-forming segment of the tracrRNA are SEQ ID NOs: 83 and 110, respectively.

It will be recognized that the dual-guide DNA-targeting RNA duplex, which comprises a crRNA and a tracrRNA molecule of the invention, can be designed to allow for controlled (i.e., conditional) binding of a crRNA with a tracrRNA. Because the DNA-targeting RNA duplex is not functional unless both the crRNA and the tracrRNA are bound in a functional complex with a site-directed modifying polypeptide, such as Cas9, a DNA-targeting RNA duplex can be inducible (e.g., drug inducible) by rendering the binding between the crRNA and the tracrRNA to be inducible. As one non-limiting example, RNA aptamers can be used to regulate (i.e., control) the binding of the crRNA with the tracrRNA. Accordingly, the crRNA and/or the tracrRNA can comprise an RNA aptamer sequence.

The protein-binding segment, which is a part of a DNA-targeting RNA duplex and comprises the duplex-forming segments of the crRNA and tracrRNA molecules, can have a length of from about 10 nucleotides to about 100 nucleotides. For example, the protein-binding segment can have a length of from about 12 nucleotides (nt) to about 80 nt, from about 12 nt to about 50 nt, from about 12 nt to about 40 nt, from about 12 nt to about 30 nt, from about 12 nt to about 25 nt, or from about 12 to about 20 nt. The dsRNA duplex of the protein-binding segment can have a length from about 6 base pairs (bp) to about 50 bp. For example, the dsRNA duplex of the protein binding segment can have a length from about 6 bp to about 40 bp, from about 6 bp to about 30 bp, from about 6 bp to about 25 bp, from about 6 bp to about 20 bp, from about 6 bp to about 15 bp, from about 8 bp to about 40 bp, from about 8 bp to about 30 bp, from about 8 bp to about 25 bp, from about 8 bp to about 20 bp or from about 8 bp to about 15 bp. For example, the dsRNA duplex of the protein-binding segment can have a length from about from about 8 bp to about 10 bp, from about 10 bp to about 15 bp, from about 15 bp to about 18 bp, from about 18 bp to about 20 bp, from about 20 bp to about 25 bp, from about 25 bp to about 30 bp, from about 30 bp to about 35 bp, from about 35 bp to about 40 bp, or from about 40 bp to about 50 bp. In some embodiments, the dsRNA duplex of the protein-binding segment has a length of 17 base pairs.

The percent complementarity between the nucleotide sequences that hybridize to form the dsRNA duplex of the protein-binding segment can be at least about 60%. For example, the percent complementarity between the nucleotide sequences that hybridize to form the dsRNA duplex of the protein-binding segment can be at least about 65%, at least about 70%, at least about 75%, at least about 80%, at least about 85%, at least about 90%, at least about 95%, at least about 98%, or at least about 99%. In some cases, the percent complementarity between the nucleotide sequences that hybridize to form the dsRNA duplex of the protein-binding segment is 100%.

The base pairing between the duplex-forming segments of crRNA and tracrRNA molecules is not 100% in the native DNA-targeting RNA duplex, as there is at least one small loop that forms (Jinek et al., 2012; Briner et al., 2014. Molecular Cell 56: 333-339) Similarly, the base pairing between the duplex-forming segments of the crRNA and tracrRNA molecules of the invention may not be 100%. In some embodiments, the protein-binding segment of a DNA-targeting RNA duplex of the invention comprises at least 8 basepairings. In other embodiments, the protein-binding segment of a DNA-targeting RNA duplex of the invention comprises at least 9 basepairings, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 basepairings.

In some embodiments, a DNA-targeting RNA duplex of the invention and a site-directed modifying polypeptide form a complex. The DNA-targeting RNA duplex provides target specificity to the complex by comprising a nucleotide sequence on the crRNA molecule that is complementary to a sequence of a target DNA (as noted above). The site-directed modifying polypeptide of the complex provides the site-specific activity. In other words, the site-directed modifying polypeptide is guided to a DNA sequence (e.g. a chromosomal sequence or an extrachromosomal sequence, e.g. an episomal sequence, a minicircle sequence, a mitochondrial sequence, a chloroplast sequence, etc.) by virtue of its association with at least the protein-binding segment of the DNA-targeting RNA duplex. A site-directed modifying polypeptide modifies the target DNA (e.g., cleavage or methylation of target DNA) and/or a polypeptide associated with target DNA (e.g., methylation or acetylation of a histone tail). A site-directed modifying polypeptide is also referred to herein as a “site-directed polypeptide” or an “RNA binding site-directed modifying polypeptide.”

In some cases, the site-directed modifying polypeptide is a naturally-occurring modifying polypeptide. In other cases, the site-directed modifying polypeptide is not a naturally-occurring polypeptide (e.g., a chimeric polypeptide or a naturally-occurring polypeptide that is modified, e.g., mutation, deletion, insertion). Exemplary naturally-occurring site-directed modifying polypeptides are known in the art (see for example, Makarova et al., 2017, Cell 168: 328-328.e1, and Shmakov et al., 2017, Nat Rev Microbiol 15(3): 169-182, both herein incorporated by reference). These naturally occurring polypeptides bind a DNA-targeting RNA, are thereby directed to a specific sequence within a target DNA, and cleave the target DNA to generate a double strand break.

A site-directed modifying polypeptide comprises two portions, an RNA-binding portion and an activity portion. In some embodiments, the site-directed modifying polypeptide comprises: (i) an RNA-binding portion that interacts with a DNA-targeting RNA, wherein the DNA-targeting RNA comprises a nucleotide sequence that is complementary to a sequence in a target DNA; and (ii) an activity portion that exhibits site-directed enzymatic activity (e.g., activity for DNA methylation, activity for DNA cleavage, activity for histone acetylation, activity for histone methylation, etc.), wherein the site of enzymatic activity is determined by the DNA-targeting RNA. In other embodiments, a site-directed modifying polypeptide comprises: (i) an RNA-binding portion that interacts with a DNA-targeting RNA, wherein the DNA-targeting RNA comprises a nucleotide sequence that is complementary to a sequence in a target DNA; and (ii) an activity portion that modulates transcription within the target DNA (e.g., to increase or decrease transcription), wherein the site of modulated transcription within the target DNA is determined by the DNA-targeting RNA.

In some cases, the site-directed modifying polypeptide has enzymatic activity that modifies target DNA (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity). In other cases, the site-directed modifying polypeptide has enzymatic activity that modifies a polypeptide (e.g., a histone) associated with target DNA (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity).

In some cases, different site-directed modifying polypeptides, for example different Cas9 proteins (i.e., Cas9 proteins from various species) may be advantageous to use in the various provided methods of the invention to capitalize on various enzymatic characteristics of the different Cas9 proteins (e.g., for different PAM sequence preferences; for increased or decreased enzymatic activity; for an increased or decreased level of cellular toxicity; to change the balance between NHEJ, homology-directed repair, single strand breaks, double strand breaks, etc.). Cas9 proteins from various species (for example, those disclosed in Shmakov et al., 2017, or polypeptides derived therefrom) may require different PAM sequences in the target DNA. Thus, for a particular Cas9 enzyme of choice, the PAM sequence requirement may be different than the 5′-N GG-3′ sequence (where N is either a A, T, C, or G) known to be required for Cas9 activity. Many Cas9 orthologues from a wide variety of species have been identified herein and the proteins share only a few identical amino acids. All identified Cas9 orthologs have the same domain architecture with a central HNH endonuclease domain and a split RuvC/RNaseH domain. Cas9 proteins share 4 key motifs with a conserved architecture; Motifs 1, 2, and 4 are RuvC like motifs, while motif 3 is an HNH-motif.

The site-directed modifying polypeptide may also be a chimeric and modified Cas9 nuclease. For example, it may be a modified Cas9 “base editor”. Base editing enables direct, irreversible conversion of one target DNA base into another in a programmable manner, without requiring DNA cleavage or a donor DNA molecule. For example, Komor et al (2016, Nature, 533: 420-424), teach a Cas9-cytidine deaminase fusion, where the Cas9 has also been engineered to be inactivated and not induce double-stranded DNA breaks. Additionally, Gaudelli et al (2017, Nature, doi:10.1038/nature24644) teach a catalytically impaired Cas9 fused to a tRNA adenosine deaminase, which can mediate conversion of an A/T to G/C in a target DNA sequence. Another class of engineered Cas9 nucleases which may act as a site-directed modifying polypeptide in the methods and compositions of the invention are variants which can recognize a broad range of PAM sequences, including NG, GAA, and GAT (Hu et al., 2018, Nature, doi:10.1038/nature26155).

Any Cas9 protein, including those naturally occurring and/or those mutated or modified from naturally occurring Cas9 proteins, can be used as a site-directed modifying polypeptide in the methods and compositions of the present invention. Catalytically active Cas9 nucleases cleave target DNA to produce double strand breaks. These breaks are then repaired by the cell in one of two ways: non-homologous end joining, and homology-directed repair.

In non-homologous end joining (NHEJ), the double-strand breaks are repaired by direct ligation of the break ends to one another. As such, no new nucleic acid material is inserted into the site, although some nucleic acid material may be lost, resulting in a deletion. In homology-directed repair, a donor DNA molecule with homology to the cleaved target DNA sequence is used as a template for repair of the cleaved target DNA sequence, resulting in the transfer of genetic information from the donor polynucleotide to the target DNA. As such, new nucleic acid material may be inserted/copied into the site. In some cases, a target DNA is contacted with a donor molecule, for example a donor DNA molecule. In some cases, a donor DNA molecule is introduced into a cell. In some cases, at least a segment of a donor DNA molecule integrates into the genome of the cell.

The modifications of the target DNA due to NHEJ and/or homology-directed repair lead to, for example, gene correction, gene replacement, gene tagging, transgene insertion, nucleotide deletion, gene disruption, gene mutation, etc. Accordingly, cleavage of DNA by a site-directed modifying polypeptide may be used to delete nucleic acid material from a target DNA sequence (e.g., to disrupt a gene that makes cells susceptible to infection (e.g. the CCR5 or CXCR4 gene, which makes T cells susceptible to HIV infection), to remove disease-causing trinucleotide repeat sequences in neurons, to create gene knockouts and mutations as disease models in research, etc.) by cleaving the target DNA sequence and allowing the cell to repair the sequence in the absence of an exogenously provided donor polynucleotide. Thus, the subject methods can be used to knock out a gene (resulting in complete lack of transcription or altered transcription) or to knock in genetic material into a locus of choice in the target DNA. Alternatively, if a DNA-targeting RNA duplex and a site-directed modifying polypeptide are co-administered to cells with a donor molecule that includes at least a segment with homology to the target DNA sequence, the subject methods may be used to add, i.e. insert or replace, nucleic acid material to a target DNA sequence (e.g. to “knock in” a nucleic acid that encodes for a protein, an siRNA, an miRNA, etc.), to add a tag (e.g., 6×His, a fluorescent protein (e.g., a green fluorescent protein; a yellow fluorescent protein, etc.), hemagglutinin (HA), FLAG, etc.), to add a regulatory sequence to a gene (e.g. promoter, polyadenylation signal, internal ribosome entry sequence (IRES), 2A peptide, start codon, stop codon, splice signal, localization signal, etc.), to modify a nucleic acid sequence (e.g., introduce a mutation), and the like. As such, a complex comprising a DNA-targeting RNA duplex and a site-directed modifying polypeptide is useful in any in vitro or in vivo application in which it is desirable to modify DNA in a site-specific, i.e. “targeted”, way, for example gene knock-out, gene knock-in, gene editing, gene tagging, etc., as used in, for example, gene therapy, e.g. to treat a disease or as an antiviral, antipathogenic, or anticancer therapeutic, the production of genetically modified organisms in agriculture, the large scale production of proteins by cells for therapeutic, diagnostic, or research purposes, the induction of iPS cells, biological research, the targeting of genes of pathogens for deletion or replacement, etc.

In some embodiments, a crRNA and/or tracrRNA of the invention comprises one or more modifications, e.g., a base modification, a backbone modification, etc, to provide the nucleic acid with a new or enhanced feature (e.g., improved stability). As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally suitable. In addition, linear compounds may have internal nucleotide base complementarity and may therefore fold in a manner to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Examples of suitable nucleic acids containing modifications include nucleic acids containing modified backbones or non-natural internucleoside linkages. Nucleic acids having modified backbones include those that retain a phosphorus atom in the backbone and those that do not have a phosphorus atom in the backbone. A crRNA or tracrRNA molecule of the invention may be a nucleic acid mimetic. The term “mimetic” as it is applied to polynucleotides is intended to include polynucleotides wherein only the furanose ring or both the furanose ring and the internucleotide linkage are replaced with non-furanose groups, replacement of only the furanose ring is also referred to in the art as being a sugar surrogate. The heterocyclic base moiety or a modified heterocyclic base moiety is maintained for hybridization with an appropriate target nucleic acid. One polynucleotide mimetic that has been reported to have excellent hybridization properties is a peptide nucleic acid (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative U.S. patents that describe the preparation of PNA compounds include, but are not limited to: U.S. Pat. Nos. 5,539,082; 5,714,331; and 5,719,262.

A crRNA or tracrRNA molecule of the invention may include one or more substituted sugar moieties. A crRNA or tracrRNA of the invention may also include nucleobase (often referred to in the art simply as “base”) modifications or substitutions. Another possible modification of a crRNA or tracrRNA molecule of the invention involves chemically linking to the polynucleotide one or more moieties or conjugates which enhance the activity, cellular distribution or cellular uptake of the oligonucleotide. A conjugate may include a “Protein Transduction Domain” or PTD (also known as a CPP-cell penetrating peptide), which may refer to a polypeptide, polynucleotide, carbohydrate, or organic or inorganic compound that facilitates traversing a lipid bilayer, micelle, cell membrane, organelle membrane, or vesicle membrane.

In some embodiments of the invention, the crRNA and the tracrRNA are components, or segments, of a longer RNA molecule, which is a non-naturally occurring, heterologous RNA molecule comprising two or more tRNA cleavage sequences. These tRNA cleavage sequences are for RNA-nucleolytic activity such as in pre-tRNA splicing, 3′end pre-mRNA endonuclease activity, pre-tRNA cleavage activity, and/or the pre-ribosomal RNA cleavage activity. When this RNA molecule is present in a cell, the native tRNA processing system cleaves the RNA molecule at the tRNA sequences. This cleaving then releases the crRNA and tracrRNA molecules into individual molecules, which then can interact to form a DNA-targeting RNA duplex. In some embodiments, the longer RNA comprises multiple copies of a crRNA and/or a tracrRNA. These multiple copies of the crRNA may contain the same protospacer sequence. In other embodiments, the longer RNA molecule contains multiple copies of different crRNA molecules, which contain different protospacer sequences to different DNA targets. The different DNA targets may be to different regions of the same gene, or to different genes. In some embodiments, the multiple crRNA molecules may contain different protospacer sequences, but each may have the same corresponding tracrRNA. In other words, the duplex-forming segment of each crRNA is the same, so that the duplex-forming segment of the corresponding tracrRNA is the same. In other embodiments, the longer RNA molecule may contain different variants of crRNA and/or tracrRNA molecules, for example a d9 tracrRNA and a d9+d11 tracrRNA (as described in FIG. 1 and in the examples).

A tRNA cleavage sequence includes any sequence and/or structural motif that actively interacts with and is cleaved by a cell's endogenous tRNA system such as RNase P, RNase Z and RNase E (bacteria). This can include structural recognition elements such as the acceptor stem, D-loop arm, T Psi C loop as well as specific sequence motifs. There are numerous tRNA active sequences and motifs known and available to those of skill in the art through sources such as the tRNA-SE program available at world wide web lowelab.ucsc.edu/tRNAscan-SE or world wide web trna.bioinf.uni-leipzig.de/DataOutput/Organisms (for all organisms), or world wide web at plantrna.ibmp.cnrs.fr/plantrna (for plants). Numerous articles and Genbank resources are also available.

The general characteristics of a tRNA are well known to the person skilled in the art. Preferably, a tRNA is formed from a single ribonucleotide molecule which is capable of folding to adopt a characteristic, “cloverleaf” secondary structure. This characteristic secondary structure comprises: (i) an acceptor stem composed of the first 7 ribonucleotides of the 5′ end of the ribonucleotide chain and the 7 ribonucleotides that precede the last 4 ribonucleotides of the 3′ end of the ribonucleotide chain, thus forming a double stranded structure comprising 6 or 7 pairs of ribonucleotides, it being possible for the ribonucleotides constituted by the first ribonucleotide of the 5′ end of the ribonucleotide chain and the ribonucleotide that precedes the last 4 ribonucleotides of the 3′ end of the ribonucleotide chain not to be paired; ii) a D arm constituted by 4 pairs of ribonucleotides and a D loop constituted by 8 to ribonucleotides, formed by the folding of a part of the ribonucleotide chain that follows the first 7 ribonucleotides of the 5′ end of the ribonucleotide chain; (iii) a stem of the anticodon constituted by 5 pairs of ribonucleotides, and a loop of the anticodon constituted by 7 ribonucleotides (stem-loop of the anticodon), formed by the folding of a part of the ribonucleotide chain that follows the D arm and the D loop; (iv) a variable loop constituted by from 4 to 21 ribonucleotides and formed by a part of the ribonucleotide chain that follows the stem of the anti codon and the loop of the anticodon; (v) a T arm constituted by 5 pairs of ribonucleotides, and a T loop constituted by 8 ribonucleotides, formed by the folding of a part of the ribonucleotide chain that follows the variable loop and precedes the ribonucleotides of the 3′ end of the ribonucleotide chain which are involved in the constitution of the acceptor stem.

In some instances, preferably, from the 5′ end in the direction towards the 3′ end, 2 ribonucleotides are present between the first 7 ribonucleotides of the 5′ end of the ribonucleotide chain and the D arm and loop, 1 ribonucleotide is present between the Darm and loop, on the one hand, and the stem and the loop of the anti-codon, on the other hand, and 1 ribonucleotide is present between the stem and the loop of the anticodon, on the one hand, and the variable loop, on the other hand. Still preferably, and according to the numbering well-known to the person skilled in the art and defined by Sprinzl et al., 1998, (Nucleic Acids Res. 26: 148-153) the tRNA comprises 17 ribonucleotides, ensuring the three-dimensional structure of the tRNA and recognition by the cell enzymes, namely: U.sub.8, A.sub.14, (A or G).sub.15, G.sub. 18, G.sub.19, A.sub.21, G.sub.53, U.sub.54, U.sub.55, C.sub. 56, (A or G).sub.57, A.sub.58, (C or U).sub.60, C.sub.61, C.sub.74, C.sub.75, A.sub.76. The indicated ribonucleotides correspond to the sequence of the tRNA as transcribed before any post-transcriptional modifications of certain ribonucleotides by the cell machinery.

In particular, the tRNA defined above may be selected from the group constituted by Archean, bacterial, viral, protozoan, fungal, algal, plant or animal tRNAs. The tRNAs which can be used according to the invention also include all the tRNAs described by Sprinzl et al. (1998) or those available on the world wide web at, for example uni-bayreuth.de/departments/biochemie/tma/. In the context of the invention, the term “tRNA” also includes structures obtained by modifying a tRNA as defined above or natural variants of a tRNA as defined above, provided that those modified structures or those variants retain the functionalities of the unmodified tRNA, namely especially the interaction with proteins such as EF-Tu′ factor (see, for example, Rodnina et al., (2005) FEBS. Lett. 579: 938-942) or CCAse (see, for example, Augustin et al. (2003) J. Mol. Biol. 328: 985-994). There are numerous tRNA active sequences and motifs known and available to those of skill in the art, for example through sources such as the tRNA-SE program or from the world wide web at plantrna.ibmp.cnrs.fr/plantrna (for plants).

As used herein, the phrase “a substrate for a 3′ end pre-mRNA endonuclease” refers to any nucleotide sequence recognized and excised by a 3′ end pre-mRNA endonuclease. For example, a nucleotide sequence comprising a hexanucleotide with the sequence AACAAA upstream and a G/U-rich sequence element downstream of the cleavage site may be utilized as a substrate for 3′ end pre-mRNA endonuclease. A nucleotide sequence recognized and excised by a 3′ end pre-mRNA endonuclease may comprise 10 nucleotides, 15 nucleotides, 20 nucleotides, 25 nucleotides, 25 nucleotides, 30 nucleotides, 40 nucleotides, 45 nucleotides, 50 nucleotides, 55 nucleotides, 60 nucleotides, 65 nucleotides, 75 nucleotides, 100 nucleotides, 125 nucleotides, 150 nucleotides, or more.

The present invention provides a DNA-targeting RNA duplex which comprises a crRNA molecule and its corresponding tracrRNA molecule, wherein the crRNA molecule and the tracrRNA molecule comprise the nucleic acid sequences of, respectively, SEQ ID NO: 55 and 56, SEQ ID NO: 57 and 58, SEQ ID NO: 59 and 60, SEQ ID NO: 61 and 62, SEQ ID NO: 63 and 64, SEQ ID NO: 65 and 66, SEQ ID NO: 67 and 68, SEQ ID NO: 69 and 70, SEQ ID NO: 71 and 72, SEQ ID NO: 73 and 74, SEQ ID NO: 75 and 76, SEQ ID NO: 77 and 78, SEQ ID NO: 79 and 80, SEQ ID NO: 81 and 82, or SEQ ID NO: 83 and 84, wherein the crRNA further comprises a nucleic acid sequence that is complementary to a sequence in a target DNA molecule, whereby the DNA-targeted RNA duplex targets and hybridizes with the target DNA sequence. The crRNA and corresponding tracrRNA molecules of the invention are engineered, meaning that they were created by the hand of man and are not naturally occurring.

A crRNA molecule of the invention comprises a protospacer sequence, which is the DNA-targeting segment of the crRNA molecule of the invention and is complementary to a sequence in a target DNA molecule. As described above, the protospacer sequence may be at least 12 nucleotides in length, with at least 90%, at least 91%, at least 92%, at least 93%, at least 94%, at least 95%, at least 96%, at least 97%, at least 98%, at least 99%, or 100% complementarity to the target sequence of the target DNA molecule. It will be appreciated by one of skill in the art that the protospacer sequence can be engineered to be any of a vast number target sequence. It will also be appreciated by one skilled in the art that the sequence of the DNA-targeting segment of the crRNA does not affect the ability of the protein-binding segment of the crRNA to hybridize with its corresponding tracrRNA. Although a crRNA molecule of the invention requires a protospacer sequence to be completely functional, the present invention does not limit the sequence or the length of the protospacer sequence beyond what is already required for proper CRISPR-Cas system targeted, as described above.

In other embodiments, the present invention provides for a DNA-targeting RNA duplex as described, wherein the duplex-forming segments of the crRNA molecule and its corresponding tracrRNA molecule comprise the nucleic acid sequences of, respectively, SEQ ID NO: 55 and 96, SEQ ID NO: 57 and 97, SEQ ID NO: 59 and 98, SEQ ID NO: 61 and 99, SEQ ID NO: 63 and 100, SEQ ID NO: 65 and 101, SEQ ID NO: 67 and 102, SEQ ID NO: 69 and 103, SEQ ID NO: 71 and 104, SEQ ID NO: 73 and 105, SEQ ID NO: 75 and 106, SEQ ID NO: 77 and 107, SEQ ID NO: 79 and 108, SEQ ID NO: 81 and 109, or SEQ ID NO: 83 and 110.

The present invention also provides a nucleic acid molecule comprising a nucleic acid sequence encoding at least one crRNA and/or at least one tracrRNA of the invention. The nucleic acid molecule may encode for more than one crRNA molecule, wherein the multiple crRNA molecules have different protospacer sequences. Alternatively, the nucleic acid molecule may encode for multiple crRNA molecules which have the same protospacer sequence. The nucleic acid molecule may also encode for multiple tracrRNA molecules, or it may encode for a single tracrRNA molecule multiple times. The nucleic acid molecule may be a DNA or an RNA molecule. In some embodiments, the nucleic acid molecule is circularized. In other embodiments, the nucleic acid molecule is linear. In some embodiments, the nucleic acid molecule is single stranded, partially double-stranded, or double-stranded.

In some embodiments, the nucleic acid molecule is complexed with at least one polypeptide. The polypeptide may have a nucleic acid recognition or nucleic acid binding domain. In some embodiments, the polypeptide is a shuttle for mediating delivery of, for example, a crRNA, a tracrRNA, and/or a DNA-targeting RNA duplex, and also a site-directed modifying polypeptide, and optionally a donor molecule. In some embodiments, the polypeptide shuttle is a Feldan Shuttle (U.S. Patent Publication No. 20160298078, herein incorporated by reference).

The nucleic acid molecule of the invention may comprise an expression cassette capable of driving the expression of at least one crRNA and/or at least one tracrRNA. The nucleic acid molecule may further comprise additional expression cassettes, capable of expressing, for example, a nuclease such as a CRISPR-associated nuclease, for example a Cas9 nuclease, a chimeric enzyme comprising part of a Cas9 nuclease, or a modified Cas9 nuclease, all of which are described above.

The present invention also provides an engineered, non-naturally occurring system for targeted mutagenesis comprising a DNA-targeting RNA duplex of the invention and a site-directed modifying polypeptide, wherein the DNA-targeting RNA duplex comprises a crRNA molecule and its corresponding tracrRNA molecule, wherein the crRNA molecule and the tracrRNA molecule comprise the nucleic acid sequences of, respectively, SEQ ID NO: 55 and 56, SEQ ID NO: 57 and 58, SEQ ID NO: 59 and 60, SEQ ID NO: 61 and 62, SEQ ID NO: 63 and 64, SEQ ID NO: 65 and 66, SEQ ID NO: 67 and 68, SEQ ID NO: 69 and 70, SEQ ID NO: 71 and 72, SEQ ID NO: 73 and 74, SEQ ID NO: 75 and 76, SEQ ID NO: 77 and 78, SEQ ID NO: 79 and 80, SEQ ID NO: 81 and 82, or SEQ ID NO: 83 and 84, wherein the crRNA further comprises a nucleic acid sequence that is complementary to a sequence in a target DNA molecule, whereby the crRNA-tracrRNA dual guide complex targets and hybridizes with the target DNA sequence, and the site-directed modifying polypeptide cleaves the DNA molecule. As described above, the crRNA molecule of the invention comprises a protospacer sequence, which is the DNA-targeting segment of the crRNA molecule of the invention and is complementary to a sequence in a target DNA molecule. As described above, the protospacer sequence may be at least 12 nucleotides in length, with at least 90%, at least 91%, at least 92%, at least 93%, at least 94%, at least 95%, at least 96%, at least 97%, at least 98%, at least 99%, or 100% complementarity to the target sequence of the target DNA molecule.

In some embodiments, the crRNA molecule, its corresponding tracrRNA molecule, and the site-directed modifying polypeptide are encoded within at least one nucleic acid molecule, wherein the crRNA molecule and the tracrRNA molecule are encoded by nucleic acid sequences comprising, respectively, SEQ ID NO: 3 and 4, SEQ ID NO: 5 and 6, SEQ ID NO: 7 and 8, SEQ ID NO: 9 and 10, SEQ ID NO: 11 and 12, SEQ ID NO: 13 and 14, SEQ ID NO: 15 and 16, SEQ ID NO: 17 and 18, SEQ ID NO: 19 and 20, SEQ ID NO: 21 and 22, SEQ ID NO: 23 and 24, SEQ ID NO: 28 and 29, SEQ ID NO: 30 and 31, SEQ ID NO: 32 and 33, or SEQ ID NO: 34 and 35, or the complements thereof, or the crRNA molecule comprises the nucleic acid sequence of SEQ ID NO: 36 or the complement thereof, wherein the crRNA further comprises a nucleic acid sequence that is complementary to a sequence in a target DNA molecule, whereby the crRNA-tracrRNA dual guide complex targets and hybridizes with the target DNA sequence, and the site-directed modifying polypeptide cleaves the DNA molecule. As described above, the protospacer sequence may be at least 12 nucleotides in length, with at least 90%, at least 91%, at least 92%, at least 93%, at least 94%, at least 95%, at least 96%, at least 97%, at least 98%, at least 99%, or 100% complementarity to the target sequence of the target DNA molecule.

In some embodiments, the nucleic acid molecule comprises at least one expression cassette which comprises a promoter driven by a RNA polymerase II. In further embodiments, the nucleic acid sequence of the promoter driven by a RNA polymerase II is at least 90% identical to SEQ ID NO: 85. In some embodiments, the nucleic acid molecule comprises at least one expression cassette which comprises a promoter driven by a RNA polymerase III. In further embodiments, the nucleic acid sequence of the promoter driven by a RNA polymerase III is at least 90% identical to SEQ ID NO: 86. In some embodiments, the nucleic acid molecule of the invention comprises at least two expression cassettes, one of which comprises a promoter driven by a RNA polymerase II and the other of which comprises a promoter driven by a RNA polymerase III. In further embodiments, the first expression cassette comprises a promoter at least 90% identical to SEQ ID NO: 85 and the second expression cassette comprises a promoter at least 90% identical to SEQ ID NO: 86.

In some embodiments, the nucleic acid molecule of the invention described above comprises two or more expression cassettes, each of which encode for the same crRNA and corresponding tracrRNA molecules. In other embodiments, the nucleic acid molecule described above comprises two or more expression cassettes, each of which encode for crRNA molecules of the invention which have differing protospacer sequences.

In some embodiments, the nucleic acid molecule is a vector. In further embodiments, the nucleic acid molecule is a vector capable of transformation, for example biolistic transformation or Agrobacterium-mediated transformation. In some embodiments, the site-directed modifying polypeptide is encoded on the same nucleic acid molecule on which the crRNA and tracrRNA molecules are encoded. In other embodiments, the site-directed modifying polypeptide is encoded on a different nucleic acid molecule from that which encodes the crRNA and tracrRNA molecules. In some embodiments, the crRNA and the tracrRNA molecules are encoded in different expression cassettes. In other embodiments, the crRNA and the tracrRNA molecules are encoded in the same expression cassette.

The present invention also provides a RNA molecule comprising at least one crRNA segment and at least one of its corresponding tracrRNA segment, wherein the segments are operably linked at the 5′ and/or 3′ end to a tRNA cleavage sequence. In some embodiments, the RNA molecule may be present in a cell capable of tRNA cleavage. Following tRNA cleavage, the crRNA segment becomes a crRNA molecule of the invention, and the tracrRNA segment becomes a tracrRNA molecule of the invention, so that the crRNA and tracrRNA molecules are separate and distinct molecules which are capable of forming a DNA-targeting RNA duplex. In some embodiments, the RNA molecule comprises a tRNA-crRNA-tRNA-tracrRNA in tandem alignment. In some embodiments, at least one of the resulting crRNA molecules and its corresponding tracrRNA molecule comprise the nucleic acid sequences of, respectively, SEQ ID NO: 55 and 56, SEQ ID NO: 57 and 58, SEQ ID NO: 59 and 60, SEQ ID NO: 61 and 62, SEQ ID NO: 63 and 64, SEQ ID NO: 65 and 66, SEQ ID NO: 67 and 68, SEQ ID NO: 69 and 70, SEQ ID NO: 71 and 72, SEQ ID NO: 73 and 74, SEQ ID NO: 75 and 76, SEQ ID NO: 77 and 78, SEQ ID NO: 79 and 80, SEQ ID NO: 81 and 82, or SEQ ID NO: 83 and 84, wherein the crRNA further comprises a nucleic acid sequence that is complementary to a sequence in a target DNA sequence. As previously stated, a crRNA molecule of the invention comprises a protospacer sequence, which is the DNA-targeting segment of the crRNA molecule of the invention and is complementary to a sequence in a target DNA molecule. As described above, the protospacer sequence may be at least 12 nucleotides in length, with at least 90%, at least 91%, at least 92%, at least 93%, at least 94%, at least 95%, at least 96%, at least 97%, at least 98%, at least 99%, or 100% complementarity to the target sequence of the target DNA molecule.

In some embodiments, the RNA molecule described above, which comprises at least one crRNA, at least one tracrRNA, and at least one tRNA cleavage sequence, contains a nucleic acid sequence of the tRNA cleavage site which is at least 90% identical to SEQ ID NO: 112.

The present invention also provides a nucleic acid molecule comprising at least one expression cassette which expresses the RNA molecule comprising tRNA cleavage sites described above. The nucleic acid molecule may be present in a cell capable of tRNA cleavage. In some embodiments, a crRNA molecule of the invention, the corresponding tracrRNA molecule of the invention, and at least two tRNA cleavage sequences are encoded within the same expression cassette, whereby following tRNA cleavage the crRNA and the tracrRNA molecules are separate and distinct molecules.

In some embodiments, the nucleic acid molecule which expresses an RNA molecule comprising at least one tRNA cleavage site comprises at least one expression cassette which comprises a promoter driven by a RNA polymerase II. In further embodiments, the promoter driven by a RNA polymerase II is at least 90% identical to SEQ ID NO: 85. In some embodiments, the nucleic acid molecule comprises at least one expression cassette which comprises a promoter driven by a RNA polymerase III. In further embodiments, the promoter driven by a RNA polymerase III is at least 90% identical to SEQ ID NO: 86. In some embodiments, the nucleic acid molecule of the invention comprises at least two expression cassettes, one of which comprises a promoter driven by a RNA polymerase II and the other of which comprises a promoter driven by a RNA polymerase III. In further embodiments, the first expression cassette comprises a promoter at least 90% identical to SEQ ID NO: 85 and the second expression cassette comprises a promoter at least 90% identical to SEQ ID NO: 86.

In some embodiments, the nucleic acid molecule of the invention described directly above comprises two or more expression cassettes, each of which may encode for the same crRNA and corresponding tracrRNA molecules. In other embodiments, the nucleic acid molecule described above comprises two or more expression cassettes, each of which may encode for crRNA molecules of the invention which have differing protospacer sequences.

In some embodiments, the nucleic acid molecule described directly above is a vector. In further embodiments, the nucleic acid molecule is a vector capable of transformation, for example biolistic transformation or Agrobacterium-mediated transformation. In some embodiments, the site-directed modifying polypeptide is encoded on the same nucleic acid molecule on which the crRNA and tracrRNA molecules are encoded. In other embodiments, the site-directed modifying polypeptide is encoded on a different nucleic acid molecule from that which encodes the crRNA and tracrRNA molecules. In some embodiments, the crRNA and the tracrRNA molecules are encoded on more than one expression cassettes. In other embodiments, the crRNA and the tracrRNA molecules are encoded in the same expression cassette.

In some embodiments, the nucleic acid molecule described directly above comprises at least one expression cassette, wherein the nucleic acid sequence of the expression cassette is any of SEQ ID NOs: 87-94. The twenty N's within SEQ ID NOs: 87-94 represent the protospacer of the crRNA molecules encoded within the expression cassette. As described above, the protospacer sequence may be at least 12 nucleotides in length, with at least 90%, at least 91%, at least 92%, at least 93%, at least 94%, at least 95%, at least 96%, at least 97%, at least 98%, at least 99%, or 100% complementarity to the target sequence of the target DNA molecule.

The present invention also provides a method of site-specific modification of a target DNA, the method comprising contacting the target DNA with: (i) a DNA-targeting RNA duplex, or a DNA molecule encoding the same, wherein the DNA-targeting RNA duplex is a DNA-targeting RNA duplex of the invention as described above, and (ii) a site-directed modifying polypeptide, or a DNA molecule encoding the same, wherein the site-directed modifying polypeptide comprises an RNA-binding portion that interacts with the DNA-targeting RNA, and an activity portion that exhibits site-directed enzymatic activity.

In some embodiments, the target DNA of the method is extrachromosomal. In further embodiments, the method is practiced in vitro, wherein the target DNA is extrachromosomal, such as a DNA vector or plasmid. In other embodiments, the target DNA is within a cell. In some embodiments, the cell is a eukaryotic cell. In further embodiments, the cell is a plant, algal, or fungal cell. In some embodiments, the target DNA is in an episome, mitochrondria or chloroplast. In other embodiments, the target DNA is part of a chromosome. In further embodiments, the target DNA is part of a chromosome in a cell.

In some embodiments, the method of site-specific modification requires the site-directed modifying polypeptide to have an enzymatic activity which modifies the target DNA. The enzymatic activity may be nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, or glycosylase activity, or any combination thereof. As described previously, the site-directed modifying polypeptide may be an engineered and/or chimeric enzyme.

Methods of the invention include site-specific modification of a target DNA wherein the DNA-modifying enzymatic activity is nuclease activity. The nuclease may introduce a single-strand or a double-stranded break in the target DNA. The DNA-targeting RNA duplex and/or the site-directed modifying polypeptide may contact the target DNA under conditions that are permissive for nonhomologous end joining (NHEJ) or homology-directed repair. In some embodiments, the target DNA may be modified as a result of the repair process, and not as a direct result of the enzymatic activity of the site-directed modifying polypeptide which may act only as a site-directed nuclease.

The present invention also provides a method of site-specific modification wherein the target site is modified by the insertion of a nucleic acid sequence. This sequence is provided by a donor molecule. In this method, the target DNA is contacted with: (i) a DNA-targeting RNA duplex, or a DNA molecule encoding the same, wherein the DNA-targeting RNA duplex is a DNA-targeting RNA duplex of the invention as described above; (ii) a site-directed modifying polypeptide, or a DNA molecule encoding the same, wherein the site-directed modifying polypeptide comprises an RNA-binding portion that interacts with the DNA-targeting RNA, and an activity portion that exhibits site-directed enzymatic activity; and (iii) a donor polynucleotide, wherein the donor polynucleotide, a portion of the donor polynucleotide, a copy of the donor polynucleotide, or a portion of a copy of the donor polynucleotide integrates into the target DNA.

In some embodiments, the site-directed modifying polypeptide of the methods of the invention is a CRISPR-associated nuclease. In further embodiments, the site-directed modifying polypeptide is an optionally modified Cas9 nuclease. Modified Cas9 nucleases may be chimeric, may have altered enzymatic activity, and/or may have no nuclease activity, as described above. A modified Cas9 where the nuclease activity is inactivated may be referred to as a dCas9.

The methods of the invention are methods of site-specific modification of a target DNA. In some embodiments, the modification is an insertion of modified gene sequence targeted to a gene within the genome, which is also called allele replacement. Without being bound by theory, the modified gene sequence is highly homologous to the targeted genomic site, so that the modified gene sequence could replace at least a portion of the nucleotides of the targeted genomic site by homologous recombination via homology-dependent repair following RNA-mediated targeted genomic cleavage. Allele replacement does not introduce foreign gene sequences. Allele replacement typically involves precise replacement of at least one nucleotide to modify gene functions, such as enzymatic activity or a regulatory function. In some embodiments, allele replacement can be used to replace a few nucleotides of a coding region of a gene to produce a new functional protein or enzyme variants containing one or a few new amino acid changes. For example, a glyphosate sensitive EPSPS gene allele can be converted into a glyphosate tolerant variant by changing 2 amino acids, T178I and P182A mutation using CRISPR-Cas9 mediated genome editing (Sauer N J et al., 2016, Plant Physiol. DOI:10.1104/pp. 15.01696). Allele replacement frequency is typically quite low in crop plants even with the use of site-directed nucleases to increase its frequency up to thousands of fold in comparison with background homology recombination frequency, thus making its application in crop improvement limited.

In some embodiments of the methods of the invention, a nucleic acid molecule encoding for an anti-silencing protein, or the anti-silencing protein itself, is also introduced into the cell which comprises the target DNA. In some embodiments, the anti-silencing protein is or is derived from a viral silencing suppressor (VSR). In further embodiments, the anti-silencing protein is a VSR derived from a plant virus. In further embodiments, the anti-silencing protein is the viral silencing suppressor p19 protein, derived from a Tombus virus, for example CymRSV, CIRV, or TBSV. Zhu et al recently showed that p19 VSR derived from Tomato Bushy Stunt Virus co-expressed with a guide RNA and a Cas9 nucleease improved gene targeting efficiency and/or guide RNA stability in plants (U.S. Patent Publication No. 2016/0264982). In some embodiments, the VSR is selected from the group of plant virus proteins including HC-Pro, p14, p38, NSs, NS3, CaMV P6, PNS10, P122, 2b, Potex p25, ToRSV CP, P0, and SPMMV P1 (see Csorba et al., 2015, Virology 479-480 p. 85-103, hereby incorporated by reference).

In some embodiments of the methods of the invention, the cell which comprises the target DNA is a monocotyledonous plant cell. In further embodiments, the plant cell is a maize, rice, sorghum, sugarcane, barley, wheat, oat, turf grass, or ornamental grass cell. In other embodiments, the cell is a dicotyledonous plant cell. In further embodiments, the plant cell is a tobacco, tomato, pepper, eggplant, sunflower, crucifer, flax, potato, cotton, soybean, sugar beet, or oilseed rape cell. In some embodiments, the cell is a coniferous cell.

In some embodiments of the methods of the invention, DNA molecules encoding for the DNA-targeting RNA duplex and/or site-directed modifying polypeptide are introduced or delivered into the cell comprising the target DNA. In some embodiments, the DNA-targeting RNA duplex and the site-directed modifying polypeptide are encoded on the same DNA molecule. In other embodiments, they are encoded on separate DNA molecules. In further embodiments, the DNA molecule(s) are introduced into the cell by biolistic bombardment, Agrobacterium mediated-transformation, or any other methods known in the art. In some embodiments, the DNA molecules are transiently expressed, and do not incorporate into the genome of the cell. In some embodiments, the DNA molecules are stably transformed, and incorporate into the genome of the cell.

The present invention also provides a method of producing a plant, plant part, or progeny thereof comprising a site-specific modification of a target DNA, said method comprising regenerating a plant from a plant cell whose DNA has been modified by any of the methods of the invention described above. The present invention further provides the plant, plant part, or progeny thereof comprising a modification of its DNA which was produced by these methods.

The present invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the present invention.

### EXAMPLES

### Example 1: Improved Genome Editing Efficiency Through Mutations of crRNA and tracrRNA

This example describes mutations to the crRNA and/or the tracrRNA to improve genome editing efficiency. These mutations generally fall under four areas. The first mutations target the RNA polymerase III pause signal. These mutations generally target two areas of the dual guide RNA complex. Both of these areas comprise a “UUUU” motif in the RNA. The continuous sequence of T's in the DNA template is a pause signal for RNA polymerase III. The first poly-U motif is located downstream of the protospacer of the crRNA. Point mutations were made to disrupt this pause signal. As shown in FIG. 1, mutants d2 through d7 were produced to determine what effect disrupting this first motif would have on genome editing efficiency. “d1” is the sequence of the crRNA without a specified sequence for the protospacer (SEQ ID NO: 113) and its corresponding tracrRNA (SEQ ID NO: 114) as shown in FIG. 1. Mutations d2 through d7 contain both a mutation in the crRNA molecule and the corresponding mutation in a tracrRNA molecule so that crRNA and the tracrRNA molecules still basepair at the mutated residue. For example, d2 is a mutation from U/A in the crRNA/tracrRNA to a G/C in the crRNA/tracrRNA of the d2 mutant. The d11 mutant targets the second UUUU motif, at the 3′ end of the crRNA and the 5′end of the tracrRNA. These mutations are illustrated in FIG. 1 and provided as SEQ ID NOs: 57 to 68.

The second area for mutagenesis focused on optimizing the crRNA-tracrRNA duplex structure, by point mutations and also by inserting additional RNA elements. To test whether extending or shorting the number of basepairs in the duplex to increase RNA:RNA interaction improved mutagenesis efficiency, the d8 mutant was produced. The d8 mutation comprises a “AAUGGUUCC” motif added to the 3′ end of the crRNA to provide complementary base pairing with the native tracrRNA (SEQ ID NO: 69). Alternatively, the d9 mutant contains a deletion of the 8 nucleotide sequence “GAACCAUU” in the 5′ end of the tracrRNA molecule to eliminate the 5′ overhang of the tracrRNA (SEQ ID NO: 72). In mutant d10, 30 nucleotides of a BYDV (Barley yellow dwarf virus) 3′-UTR is introduced at the 3′ end of the crRNA (SEQ ID NO: 73) and 37 nucleotides of a BYDV 5′-UTR is introduced at the 5′end of the tracrRNA (SEQ ID NO: 74). These sequences from BYDV contain an RNA-RNA loop interaction structure which encodes a BYDV translation element that is recognized by plant host factors, and provide an additional RNA:RNA element to the DNA-targeting RNA duplex. All of these mutations are illustrated in FIG. 1.

The third area for mutagenesis focused on transcript processing of the dual-guide RNA. There are reports that introducing tRNAs into single guide RNAs designed to target multiple sites is an effective way to allow for processing of the RNA in a cell (Xie et al., 2015, PNAS 112 (11): 3570-3575; Qi et al., 2016, BMC Biotechnology, DOI 10.1186/s12896-016-0289-2). However, the ability of the tRNAs to increase efficiency in a dual-guide RNA system has not been demonstrated. Construct 23999 was produced, which contained an expression cassette (SEQ ID NO: 25) comprising a PolIII promoter, namely prOsU3, operably linked at its 3′ end to a tRNA, which was operably linked at its 3′end to a crRNA, which was operably linked at its 3′ end to a second tRNA, which was operably linked at its 3′ end to a tracrRNA, which was operably linked at its 3′ end to a polyT. This produces an RNA molecule with tRNA-crRNA-tRNA-tracrRNA in tandem alignment. Construct 24000 was also produced, which contained an expression cassette comprising the same elements as in construct 23999 but the tracrRNA was upstream of the crRNA. In other words, construct 24000 contained an expression cassette (SEQ ID NO: 26) comprising the prOsU3 promoter, operably linked at its 3′end to a tRNA, operably linked at its 3′ end to a tracrRNA, operably linked at its 3′end to a tRNA, operably linked at its 3′ end a crRNA, operably linked at its 3′end to a polyT. This produces an RNA molecule with tRNA-tracrRNA-tRNA-crRNA in tandem alignment.

### Example 2: Targeted Mutagenesis of Rice

To test the genome editing efficacy of the dual guide constructs described above, a target was identified in the genome of rice (Oryza sativa). The target gene is DENSE AND ERECT PANICLE 1 (DEP1). The Japonica rice dep1 mutant contains a 625 bp deletion close to the 3′end of DEP1. The mutant has dense and erect panicles with a higher grain number and lower plant height than wild type (Huang et al., 2009, Nat Genet 41: 494-497. Indica rice has a wild type copy of the DEP1 gene. For the examples described here, the DEP1 was targeted for mutation, and all the crRNA constructs described here contain 5′-ACTGCAGTGCGTGCTGCGC-3′ (SEQ ID NO: 45) which encodes the protospacer for the crRNA molecules described here. It will be appreciated by one of skill in the art that present invention is not limited to the sequence of the protospacer or its corresponding DNA target, and that the mutations to the crRNA and tracrRNA molecules, as well as the expression cassettes from which they are produced, can be adapted for any protospacer sequence.

All binary vectors described here comprise an expression cassette to express a Cas9 endonuclease (WO16106121, incorporated by reference in its entirety herein) and a second expression cassette to express the selectable marker for transformation. The PMI gene (also referred to as the manA gene), which encodes for the selectable marker phosphomannose isomerase and provides the ability to metabolize mannose (U.S. Pat. Nos. 5,767,378 and 5,994,629, incorporated by reference herein), was used as a selectable marker for transformation and regeneration of transgenic rice plants. For all binary vectors except 23999 and 24000, each binary vector further comprised an expression cassette which produced the wild type or d1-d11 mutants of the crRNA, and an additional expression cassette which produced the wild type or d1-d11 mutants of the corresponding tracrRNA molecules. For 23999 and 24000, a single expression cassette produced an RNA molecule comprising tRNA, crRNA, and tracrRNA, as described above. As controls, a binary vector which comprises expression cassettes to produce wild-type crRNA and tracrRNA molecules (construct 23844) and a binary vector which comprises an expression cassette that produces a single-guide RNA molecule (construct 23127) were also tested. All expression cassettes in each binary vector are part of a single transgene.

The rice (Oryza sativa) inbred line IR58025B was used for the Agrobacterium-mediated transformation experiments essentially following the protocols for transformation, selection, and regeneration as described in Gui et al. 2014 (Plant Cell Rep 33: 1081-1090, herein incorporated by reference). The transgenic rice lines were grown in a greenhouse with 16 h light/30° C. and 8 h dark/22° C.

Leaf tissue from T0 transgenic events were sampled and used for genomic DNA extraction followed by TaqMan analysis. TaqMan analysis was essentially carried out as described in Ingham et al. (Biotechniques 31(1):132-4, 136-40, 2001), herein incorporated by reference. TaqMan was performed to detect the presence of the PMI gene (SEQ ID NO: 46-47 are primers; SEQ ID NO: 48 is the probe), the Cas9 gene (SEQ ID NOs: 49-50 are the primers; SEQ ID NO: 51 is the probe), and targeted mutations in DEP1 (SEQ ID NOs: 52-53 are the primers; SEQ ID NO: 54 is the probe). To detect mutations in DEP1, the forward primer (SEQ ID NO: 52) and the reverse primer (SEQ ID NO: 53) flank the protospacer target sequence (SEQ ID NO: 45), and the probe (SEQ ID NO: 54) hybridizes to a region of the protospacer which includes the Cas9 cutting site and the PAM. If a mutation (typically an indel) is introduced at the Cas9 cutting site, the probe will not bind to the target sequence, and therefore will not generate fluorescence. The mutation rate is calculated based on the TaqMan analysis of DEP1.

Table 1 illustrates targeted mutagenesis by DNA-targeting RNA duplexes of the invention. The SEQ ID NOs. are DNA sequences which encode the mutated crRNA (not including the protospacer) and corresponding tracrRNA molecules, or the expression cassettes which encode for the RNA molecules of constructs 23999 and 24000. The “No. of explants” is the number of rice explants initially transformed, and the “No. of transformants” is the number of explants successfully transformed. The mutation rate is the percentage of transformants which contained a targeted mutation at the DEP1 as determined by the TaqMan assay described above. The “Copy No. for mutants” indicates the number of transgene insertions in the transformants which were successfully mutated in the DEP1 gene. A “low copy” suggests a single insertion. A “two copy” suggests 2 insertions, and a high copy suggests more than two insertions.

TABLE 1
DNA-targeting RNA duplex efficiency and copy number distribution in rice transgenic events

| | | | | | | Copy No. for Mutants | | |
|---|---|---|---|---|---|---|---|---|
| | Expressed RNA | SEQ ID | No. of | No. of | Mutation | Low | Two | High |
| Construct | molecules | NOs. | explants | transformants | rate (%) | copy | copy | copy |
| 23127 | sgRNA | 27 | 172 | 45 | 55.6 | 6 | 2 | 16 |
| 23844 | WT crRNA/tracrRNA | 1-2 | 144 | 31 | 3.2 | 0 | 1 | 0 |
| 24028 | d1 crRNA/tracrRNA | 3-4 | 480 | 202 | 19.3 | 1 | 3 | 35 |
| 24027 | d2 crRNA/tracrRNA | 5-6 | 542 | 119 | 3.4 | 0 | 0 | 4 |
| 24026 | d3 crRNA/tracrRNA | 7-8 | 708 | 207 | 4.3 | 0 | 0 | 11 |
| 24030 | d4 crRNA/tracrRNA | 9-10 | 652 | 196 | 7.1 | 0 | 0 | 14 |
| 24029 | d5 crRNA/tracrRNA | 11-12 | 778 | 264 | 16.3 | 1 | 2 | 40 |
| 24025 | d6 crRNA/tracrRNA | 13-14 | 654 | 283 | 16.3 | 0 | 1 | 43 |
| 24017 | d7 crRNA/tracrRNA | 15-16 | 599 | 165 | 14.5 | 0 | 0 | 24 |
| 24015 | d8 crRNA/tracrRNA | 17-18 | 440 | 197 | 0 | 0 | 0 | 0 |
| 24016 | d9 crRNA/tracrRNA | 19-20 | 519 | 180 | 50 | 4 | 9 | 77 |
| 24013 | d10 crRNA/tracrRNA | 21-22 | 540 | 291 | 2.7 | 1 | 0 | 7 |
| 24012 | d11 crRNA/tracrRNA | 23-24 | 695 | 244 | 32.4 | 3 | 10 | 66 |
| 23999 | tRNA:crRNA:tRNA:tracrRNA | 25 | 636 | 212 | 27.8 | 2 | 4 | 53 |
| 24000 | tRNA:tracrRNA:tRNA:crRNA | 26 | 495 | 157 | 56.7 | 16 | 18 | 52 |

As can be seen in Table 1, some mutations to the crRNA/tracrRNA duplex surprisingly resulted in mutation rates several-fold better than the wild-type crRNA/tracrRNA duplex. In particular, the d9 and d11 mutations, as well as the RNA molecules produced by the 23999 and 24000 constructs, gave higher mutation rates compared to the wild-type crRNA/tracrRNA duplex.

### Example 3: Further Optimization of Dual Guide RNA Molecules

Additional mutated constructs were generated to determine if the DNA-targeting RNA duplex structure could be further optimized by mutagenesis of the crRNA and/or the tracrRNA, particularly in the regions where they interact to form a duplex, or by inserting additional RNA elements. Many of these constructs build upon the results shown in Table 1.

Construct 24127 encodes for a crRNA molecule which contains the d11 mutation (SEQ ID No: 28) and a tracrRNA molecule which contains both the d9 5′-end deletion mutation and the d11 mutation described above and in FIG. 1 (SEQ ID NO 29). Construct 24128 also encodes for a tracrRNA which has the d9 deletion mutation. Additionally, the crRNA and tracrRNA of construct 24128 have been mutated to increase the GC content in 10 nucleotides of the duplex, at the 3′ end of the crRNA (see SEQ ID NO: 30) and the 5′ end of the tracer RNA (see SEQ ID NO: 31).

Construct 24141 comprises a tracrRNA which has the d9 5′-end deletion mutation described above and in FIG. 1. Additionally, a 30 nucleotide long palindromic structure has been introduced at the 3′ end of the crRNA (see SEQ ID NO: 32) and the 5′ end of the tracrRNA (see SEQ ID NO: 33) to extend the crRNA/tracrRNA duplex by 30 nucleotides. Construct 24129 includes the mutations for 24141 but a 4 nucleotide long motif (see SEQ ID NO: 34 for crRNA and SEQ ID NO: 35 for tracrRNA) is inserted within the palindromic structure, to create a loop in the crRNA/tracrRNA duplex structure within the palindrome.

Similar to construct 24127, construct 24154 encodes for a crRNA molecule which contains the d11 mutation and a tracrRNA molecule which contains both the d9 5′-end deletion mutation and the d11 mutation. Construct 24154 further comprises a wheat dwarf virus DNA replicon, so that the crRNA and tracrRNA are expressed from the WDV DNA replicon (SEQ ID NO: 36). Construct 24154 also comprises an expression cassette for RepA, the WDV replicase. Production of additional DNA molecules encoding for the crRNA and tracrRNA by the WDV replicase may increase crRNA and tracrRNA molecules in the cell, thereby increasing mutagenesis efficiency (Wang et al., 2017, Molecular Plant 10: 1007-1010; Baltes et al., 2014, Plant Cell 26: 151-163).

The fourth area for mutagenesis focused on increasing the level of dual guide RNA molecules present in the target cell. RNA Polymerase II is known to be involved in the transcription of mRNAs, and RNA Polymerase III is known to be involved in the transcription of non-coding RNAs, including tRNAs and other small RNA molecules. To determine if transcription by a different RNA polymerase may increase genome editing efficiency in the dual-guide RNA/CRISPR system, an RNA PolII promoter was used, rather than an RNA PolIII promoter. These constructs also have two copies each of the crRNA and tracrRNA to also increase the number of crRNA and tracrRNA molecules present in the cell.

Construct 24140 contained an expression cassette comprising the prOsU3 promoter, operably linked at its 3′ end to tRNA, operably linked at its 3′ end to a tracrRNA, operably linked at its 3′end to a tRNA, operably linked at its 3′ end a crRNA, operably linked at its 3′ end to a second copy of the tracrRNA, operably linked at its 3′ end to a tRNA, operably linked at its 3′end to a second copy of the crRNA, operably linked at its 3′ end to a poly-T (SEQ ID NO: 37). Construct 24155 contained an expression cassette comprising the prOsU3 promoter, operably linked at its 3′ end to tRNA, operably linked at its 3′ end to a d9 tracrRNA, operably linked at its 3′end to a tRNA, operably linked at its 3′ end a d9 crRNA, operably linked at its 3′ end to a second copy of the d9 tracrRNA, operably linked at its 3′ end to a tRNA, operably linked at its 3′ end to a second copy of the d9 crRNA, operably linked at its 3′ end to a poly-T (SEQ ID NO: 38). Construct 24155 differs construct 24140 because construct 24155 contains dual-guide RNA molecules with the “d9” mutation, whereas the construct 24140 contains wild-type dual-guide RNA molecules.

Construct 24164 contained an expression cassette comprising a prSoUbi4 promoter, which is a RNA Polymerase II dependent promoter, operably linked at its 3′ end to tRNA, operably linked at its 3′ end to a d9 tracrRNA, operably linked at its 3′ end to a tRNA, operably linked at its 3′ end a d9 crRNA, operably linked at its 3′end to a second copy of the d9 tracrRNA, operably linked at its 3′ end to a tRNA, operably linked at its 3′ end to a second copy of the d9 crRNA, operably linked at its 3′ end to a poly-T (SEQ ID NO: 40).

Construct 24165 contained two expression cassettes for expression of crRNA and tracrRNA molecules, where the crRNA and tracrRNA molecules are mutated to contain the d9 mutation. The first expression cassette comprised a prOsU3 promoter operably linked at its 3′ end to tRNA, operably linked at its 3′ end to a d9 tracrRNA, operably linked at its 3′end to a tRNA, operably linked at its 3′ end a d9 crRNA, operably linked at its 3′ end to a second copy of the d9 tracrRNA, operably linked at its 3′ end to a tRNA, operably linked at its 3′ end to a second copy of the d9 crRNA, operably linked at its 3′ end to a poly-T (SEQ ID NO: 41). The second expression cassette comprised a prSoUbi4 promoter operably linked at its 3′end to tRNA, operably linked at its 3′ end to a d9 tracrRNA, operably linked at its 3′ end to a tRNA, operably linked at its 3′ end a d9 crRNA, operably linked at its 3′ end to a second copy of the d9 tracrRNA, operably linked at its 3′ end to a tRNA, operably linked at its 3′ end to a second copy of the d9 crRNA, operably linked at its 3′ end to a poly-T (SEQ ID NO: 42).

Construct 24169 also contained two expression cassettes for expression of crRNA and tracrRNA molecules, however the crRNA and tracrRNA molecules are mutated to contain both the d9 and the d11 mutations. The first expression cassette comprised a prOsU3 promoter operably linked at its 3′end to tRNA, operably linked at its 3′ end to a d9+d11 tracrRNA, operably linked at its 3′ end to a tRNA, operably linked at its 3′ end a d9+d11 crRNA, operably linked at its 3′ end to a second copy of the d9+d11 tracrRNA, operably linked at its 3′ end to a tRNA, operably linked at its 3′end to a second copy of the d9+d11 crRNA, operably linked at its 3′ end to a poly-T (SEQ ID NO: 43). The second expression cassette comprised a prSoUbi4 promoter operably linked at its 3′end to tRNA, operably linked at its 3′ end to a d9+d11 tracrRNA, operably linked at its 3′end to a tRNA, operably linked at its 3′ end a d9+d11 crRNA, operably linked at its 3′ end to a second copy of the d9+d11 tracrRNA, operably linked at its 3′ end to a tRNA, operably linked at its 3′end to a second copy of the d9+d11 crRNA, operably linked at its 3′ end to a poly-T (SEQ ID NO: 44).

It is important to note that the constructs described above incorporate mutations and structures which in Table 1 were shown empirically to improve mutagenesis efficiency of the crRNA/tracrRNA duplex, also referred to as the DNA-targeting RNA duplex. The success of any of these constructs could not be predicted. The optimal sequence/construct could only be determined empirically.

### Example 4: Targeted Mutagenesis of Rice

To test the constructs described in Example 3, the crRNA constructs target DEP1, as described in Example 2. Binary constructs described in this example are similar to those in Example 2 and include the PMI gene and a gene encoding a Cas9 endonuclease. Transformation of rice variety IR58025B and TaqMan analysis of the transformants was performed as described in Example 2.

Table 2 illustrates targeted mutagenesis by DNA-targeting RNA duplexes of the invention. For constructs 24127, 24128, 24141, and 24129, the SEQ ID NOs. are DNA sequences which encode the mutated crRNA (not including the protospacer) and corresponding tracrRNA molecules. For construct 24154, SEQ ID NO: 36 is the DNA sequence of the WDV DNA replicon, comprising a d11 mutant of crRNA and a d9+d11 mutant of tracrRNA. For constructs 24140, 24155, 24164, 24165, and 24169 the SEQ ID NO. is a DNA sequence of the expression cassette(s), including the promoter. The “No. of explants” is the number of rice explants initially transformed, and the “No. of transformants” is the number of explants successfully transformed. The mutation rate is the percentage of transformants which contained a targeted mutation at the DEP1 as determined by the TaqMan assay described above. The “Copy No. for mutants” indicates the number of transgene insertions in the transformants which were successfully mutated in the DEP1 gene. A “low copy” suggests a single insertion. A “two copy” suggests 2 insertions, and a high copy suggests more than two insertions.

TABLE 2
DNA-targeting RNA duplex efficiency and copy number distribution in rice transgenic events

| | | | | | | Copy No. for Mutants | | |
|---|---|---|---|---|---|---|---|---|
| | | SEQ ID | No. of | No. of | Mutation | Low | Two | High |
| Construct | RNA molecule | NOs. | explants | transformants | rate (%) | copy | copy | copy |
| 24127 | d9 + d11 | 28-29 | 780 | 164 | 50.6 | 4 | 8 | 50 |
| 24128 | d9 + new mutation | 30-31 | 737 | 172 | 44.2 | 7 | 4 | 44 |
| 24141 | d9 + new mutation | 32-33 | 613 | 186 | 29.6 | 2 | 2 | 48 |
| 24129 | d9 + new mutation | 34-35 | 494 | 169 | 24.9 | 3 | 0 | 31 |
| 24154 | d9 + d11 + DNA | 36 | 952 | 159 | 8.2 | 1 | 2 | 8 |
| | replicon | | | | | | | |
| 24140 | two repeat tRNA | 37 | 633 | 201 | 23.9 | 2 | 4 | 37 |
| | cassette driven by | | | | | | | |
| | Pol III promoter | | | | | | | |
| 24155 | two repeat tRNA | 38 | 570 | 163 | 41.1 | 6 | 5 | 42 |
| | cassette + d9 driven | | | | | | | |
| | by Pol III promoter | | | | | | | |
| 24164 | two repeat tRNA | 40 | 574 | 217 | 53.9 | 13 | 6 | 68 |
| | cassette driven by | | | | | | | |
| | Pol II promoter | | | | | | | |
| 24165 | two repeat tRNA | 41-42 | 566 | 226 | 53.5 | 22 | 11 | 59 |
| | cassette + d9 driven | | | | | | | |
| | by Pol II and Pol III | | | | | | | |
| | promoter | | | | | | | |
| | respectively | | | | | | | |
| 24169 | two repeat tRNA | 43-44 | 710 | 248 | 82.3 | 21 | 15 | 120 |
| | cassette + d9 + d11 | | | | | | | |
| | driven by Pol II and | | | | | | | |
| | Pol III promoter | | | | | | | |
| | respectively | | | | | | | |

As shown in Table 2, the mutation rate with many of these constructs is quite high. Constructs which produce crRNA/tracrRNA molecules that comprise both mutations d9 and d11 (24127 and 24169) perform very well. Interestingly, the mutation rate obtained using construct 24169 is over 150% of that obtained using construct 24165, although they only differ by the presence of both mutations d9 and d11 in construct 241696 (construct 24165 comprises the d9 mutation only). The d11 mutation changes UU/AA to GG/CC in the protein-binding RNA duplex region of the crRNA and corresponding tracrRNA, respectively. It is surprising and unexpected that such a mutation, which introduces a stronger bond for two basepairings but does not change the total number of basepairings between the crRNA and tracrRNA, can significantly increase the dual guide RNA mutation efficiency.

It will be appreciated by one skilled in the art that the examples described here can be extended to any genomic target/protospacer sequence, as the protospacer sequence is not critical for the crRNA/tracrRNA duplex formation (Jinek et al., 2012), transcription by the RNA polymerase II or RNA polymerase III, or for tRNA processing.

Although the foregoing invention has been described in some detail by way of illustration and example for purposes of clarity of understanding, it will be apparent that certain changes and modifications may be practiced with the scope of the present invention.

1-50. (canceled)

51. A DNA-targeting RNA duplex which comprises a crRNA molecule and its corresponding tracrRNA molecule, wherein the crRNA molecule and the tracrRNA molecule comprise the nucleic acid sequences of, respectively, SEQ ID NO: 55 and 56, SEQ ID NO: 57 and 58, SEQ ID NO: 59 and 60, SEQ ID NO: 61 and 62, SEQ ID NO: 63 and 64, SEQ ID NO: 65 and 66, SEQ ID NO: 67 and 68, SEQ ID NO: 69 and 70, SEQ ID NO: 71 and 72, SEQ ID NO: 73 and 74, SEQ ID NO: 75 and 76, SEQ ID NO: 77 and 78, SEQ ID NO: 79 and 80, SEQ ID NO: 81 and 82, or SEQ ID NO: 83 and 84, wherein the crRNA further comprises a DNA-targeting segment which comprises a nucleic acid sequence that is complementary to a sequence in a target DNA molecule, whereby the DNA-targeted RNA duplex targets and hybridizes with the target DNA sequence.

52. The DNA-targeting RNA duplex of claim 51, wherein the DNA-targeting segment of the crRNA molecule comprises a nucleic acid sequence at least 12 nucleotides in length and with at least 80% complementarity to a sequence in a target DNA molecule.

53. The DNA-targeting RNA duplex of claim 51, wherein the duplex forming segments of the crRNA molecule and its corresponding tracrRNA molecule comprise the nucleic acid sequences of, respectively, SEQ ID NO: 55 and 96, SEQ ID NO: 57 and 97, SEQ ID NO: 59 and 98, SEQ ID NO: 61 and 99, SEQ ID NO: 63 and 100, SEQ ID NO: 65 and 101, SEQ ID NO: 67 and 102, SEQ ID NO: 69 and 103, SEQ ID NO: 71 and 104, SEQ ID NO: 73 and 105, SEQ ID NO: 75 and 106, SEQ ID NO: 77 and 107, SEQ ID NO: 79 and 108, SEQ ID NO: 81 and 109, or SEQ ID NO: 83 and 110.

54. A DNA molecule which encodes for the crRNA or the tracrRNA molecule of claim 51.

55. A DNA molecule which encodes for both the crRNA molecule and the tracrRNA molecule of claim 51.

56. The DNA molecule of claim 51, wherein the crRNA molecule and its corresponding tracrRNA molecule are encoded by nucleic acid sequences comprising, respectively, SEQ ID NO: 3 and 4, SEQ ID NO: 5 and 6, SEQ ID NO: 7 and 8, SEQ ID NO: 9 and 10, SEQ ID NO: 11 and 12, SEQ ID NO: 13 and 14, SEQ ID NO: 15 and 16, SEQ ID NO: 17 and 18, SEQ ID NO: 19 and 20, SEQ ID NO: 21 and 22, SEQ ID NO: 23 and 24, SEQ ID NO: 28 and 29, SEQ ID NO: 30 and 31, SEQ ID NO: 32 and 33, or SEQ ID NO: 34 and 35, or the complements thereof, wherein the crRNA further comprises a DNA-targeting segment which comprises a nucleic acid sequence that is complementary to a sequence in a target DNA molecule, whereby the DNA-targeted RNA duplex targets and hybridizes with the target DNA sequence.

57. An engineered, non-naturally occurring system for targeted mutagenesis comprising the DNA-targeting RNA duplex of claim 51 and further comprising a site-directed modifying polypeptide, whereby the DNA-targeting RNA duplex interacts with the site-directed modifying polypeptide to form a complex, wherein the complex targets to and hybridizes with the target DNA molecule, and the site-directed modifying polypeptide modifies the target DNA sequence.

58. The system for targeted mutagenesis of claim 57, wherein at least one crRNA molecule, at least one tracrRNA molecule, and the site-directed modifying polypeptide are encoded within at least one nucleic acid molecule, wherein at least one crRNA molecule and its corresponding tracrRNA molecule are encoded by nucleic acid sequences comprising, respectively, SEQ ID NO: 3 and 4, SEQ ID NO: 5 and 6, SEQ ID NO: 7 and 8, SEQ ID NO: 9 and 10, SEQ ID NO: 11 and 12, SEQ ID NO: 13 and 14, SEQ ID NO: 15 and 16, SEQ ID NO: 17 and 18, SEQ ID NO: 19 and 20, SEQ ID NO: 21 and 22, SEQ ID NO: 23 and 24, SEQ ID NO: 28 and 29, SEQ ID NO: 30 and 31, SEQ ID NO: 32 and 33, or SEQ ID NO: 34 and 35, or the complements thereof, wherein the crRNA further comprises a DNA-targeting segment which comprises a nucleic acid sequence that is complementary to a sequence in a target DNA sequence, whereby the DNA-targeting RNA duplex interacts with the site-directed modifying polypeptide to form a complex, wherein the complex targets to and hybridizes with the target DNA molecule, and the site-directed modifying polypeptide modifies the target DNA sequence.

59. The nucleic acid molecule of claim 58, wherein at least one crRNA molecule and at least one tracrRNA molecule are encoded within the same expression cassette, wherein the expression cassette comprises two or more tRNA cleavage sequences, whereby following tRNA cleavage the crRNA and the tracrRNA molecules are separate and distinct molecules.

60. A method of site-specific modification of a target DNA in a eukaryotic cell, the method comprising:
contacting the target DNA with:
(i) a DNA-targeting RNA duplex, or a DNA molecule encoding the same, wherein the DNA-targeting RNA duplex is a DNA-targeting RNA duplex of claim 1; and
(ii) a site-directed modifying polypeptide, or a DNA molecule encoding the same, wherein the site-directed modifying polypeptide comprises an RNA-binding portion that interacts with the DNA-targeting RNA, and an activity portion that exhibits site-directed enzymatic activity;
wherein the enzymatic activity modifies the target DNA.

61. The method of claim 60, wherein the enzymatic activity is nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, or glycosylase activity.

62. The method of claim 61, wherein the DNA-modifying enzymatic activity is nuclease activity.

63. The method of claim 60, the method further comprising contacting the target DNA molecule with a donor polynucleotide, wherein the donor polynucleotide, a portion of the donor polynucleotide, a copy of the donor polynucleotide, or a portion of a copy of the donor polynucleotide integrates into the target DNA molecule.

64. The method of claim 60, wherein the eukaryotic cell is a plant, fungal, or algal cell.

65. The method of claim 64, wherein the plant cell is a monocotyledonous plant cell.

66. The method of claim 65, wherein the plant cell is a maize, sorghum, sugarcane, barley, wheat, oat, turf grass, or ornamental grass cell.

67. The method of claim 64, wherein the plant cell is a dicotyledonous plant cell.

68. The method of claim 67, wherein the plant cell is tobacco, tomato, pepper, eggplant, sunflower, crucifer, flax, potato, cotton, soybean, sugar beet, or oilseed rape cell.


SEQUENCE LISTING (114 sequences)

SEQ ID NO: 1 - DNA, 45 bp, Artificial Sequence
  Streptococcus pyogenes
gttttagagctatgctgttttgaatggtcccaaaacttttttttt

SEQ ID NO: 2 - DNA, 88 bp, Artificial Sequence
  Streptococcus pyogenes
ggaaccattcaaaacagcatagcaagttaaaataaggctagtccgttatcaacttgaaaaagtggcaccgagtcggtgcttttttttt

SEQ ID NO: 3 - DNA, 31 bp, Artificial Sequence
  Streptococcus pyogenes
gttttagagctatgctgttttgttttttttt

SEQ ID NO: 4 - DNA, 88 bp, Artificial Sequence; identical to SEQ ID NO: 2
  Streptococcus pyogenes

SEQ ID NO: 5 - DNA, 31 bp, Artificial Sequence
  Streptococcus pyogenes
ggtttagagctatgctgttttgttttttttt

SEQ ID NO: 6 - DNA, 88 bp, Artificial Sequence
  Streptococcus pyogenes
ggaaccattcaaaacagcatagcaagttaaactaaggctagtccgttatcaacttgaaaaagtggcaccgagtcggtgcttttttttt

SEQ ID NO: 7 - DNA, 31 bp, Artificial Sequence
  Streptococcus pyogenes
gtgttagagctatgctgttttgttttttttt

SEQ ID NO: 8 - DNA, 88 bp, Artificial Sequence
  Streptococcus pyogenes
ggaaccattcaaaacagcatagcaagttaacataaggctagtccgttatcaacttgaaaaagtggcaccgagtcggtgcttttttttt

SEQ ID NO: 9 - DNA, 31 bp, Artificial Sequence
  Streptococcus pyogenes
gttgtagagctatgctgttttgttttttttt

SEQ ID NO: 10 - DNA, 88 bp, Artificial Sequence
  Streptococcus pyogenes
ggaaccattcaaaacagcatagcaagttacaataaggctagtccgttatcaacttgaaaaagtggcaccgagtcggtgcttttttttt

SEQ ID NO: 11 - DNA, 31 bp, Artificial Sequence
  Streptococcus pyogenes
gtttgagagctatgctgttttgttttttttt

SEQ ID NO: 12 - DNA, 88 bp, Artificial Sequence
  Streptococcus pyogenes
ggaaccattcaaaacagcatagcaagttcaaataaggctagtccgttatcaacttgaaaaagtggcaccgagtcggtgcttttttttt

SEQ ID NO: 13 - DNA, 31 bp, Artificial Sequence
  Streptococcus pyogenes
gtttaagagctatgctgttttgttttttttt

SEQ ID NO: 14 - DNA, 88 bp, Artificial Sequence
  Streptococcus pyogenes
ggaaccattcaaaacagcatagcaagtttaaataaggctagtccgttatcaacttgaaaaagtggcaccgagtcggtgcttttttttt

SEQ ID NO: 15 - DNA, 31 bp, Artificial Sequence
  Streptococcus pyogenes
gtttcagagctatgctgttttgttttttttt

SEQ ID NO: 16 - DNA, 88 bp, Artificial Sequence
  Streptococcus pyogenes
ggaaccattcaaaacagcatagcaagttgaaataaggctagtccgttatcaacttgaaaaagtggcaccgagtcggtgcttttttttt

SEQ ID NO: 17 - DNA, 40 bp, Artificial Sequence
  Streptococcus pyogenes
gttttagagctatgctgttttgaatggttccttttttttt

SEQ ID NO: 18 - DNA, 88 bp, Artificial Sequence; identical to SEQ ID NO: 2
  Streptococcus pyogenes

SEQ ID NO: 19 - DNA, 31 bp, Artificial Sequence; identical to SEQ ID NO: 3
  Streptococcus pyogenes

SEQ ID NO: 20 - DNA, 80 bp, Artificial Sequence
  Streptococcus pyogenes
gcaaaacagcatagcaagttaaaataaggctagtccgttatcaacttgaaaaagtggcaccgagtcggtgcttttttttt

SEQ ID NO: 21 - DNA, 61 bp, Artificial Sequence
  Streptococcus pyogenes
gttttagagctatgctgttttgagctcgggtaggctgtcaacctaccgccgtttttttttt

SEQ ID NO: 22 - DNA, 126 bp, Artificial Sequence
  Streptococcus pyogenes
gcggtccccttattgcctgacaagctgagggccaccctggaaccattcaaaacagcatagcaagttaaaataaggctagtccgttatcaacttgaaaaagtggcaccgagtcggtgcttttttttt

SEQ ID NO: 23 - DNA, 31 bp, Artificial Sequence
  Streptococcus pyogenes
gttttagagctatgctgtggtgttttttttt

SEQ ID NO: 24 - DNA, 88 bp, Artificial Sequence
  Streptococcus pyogenes
ggaaccattcaccacagcatagcaagttaaaataaggctagtccgttatcaacttgaaaaagtggcaccgagtcggtgcttttttttt

SEQ ID NO: 25 - DNA, 663 bp, Artificial Sequence
  Streptococcus pyogenes, Oryza sativa
aaggaatctttaaacatacgaacagatcacttaaagttcttctgaagcaacttaaagttatcaggcatgcatggatcttggaggaatcagatgtgcagtcagggaccatagcacaagacaggcgtcttctactggtgctaccagcaaatgctggaagccgggaacactgggtacgttggaaaccacgtgatgtgaagaagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctgatttaaaagagttgtgcagatgatccgtggcaacaaagcaccagtggtctagtggtagaatagtaccctgccacggtacagacccgggttcgattcccggctggtgcaactgcagtgcgtgctgcgcgttttagagctatgctgttttgaacaaagcaccagtggtctagtggtagaatagtaccctgccacggtacagacccgggttcgattcccggctggtgcaggaaccattcaaaacagcatagcaagttaaaataaggctagtccgttatcaacttgaaaaagtggcaccgagtcggtgcttttttttt

SEQ ID NO: 26 - DNA, 660 bp, Artificial Sequence
  Streptococcus pyogenes, Oryza sativa
aaggaatctttaaacatacgaacagatcacttaaagttcttctgaagcaacttaaagttatcaggcatgcatggatcttggaggaatcagatgtgcagtcagggaccatagcacaagacaggcgtcttctactggtgctaccagcaaatgctggaagccgggaacactgggtacgttggaaaccacgtgatgtgaagaagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctgatttaaaagagttgtgcagatgatccgtggcaacaaagcaccagtggtctagtggtagaatagtaccctgccacggtacagacccgggttcgattcccggctggtgcaggaaccattcaaaacagcatagcaagttaaaataaggctagtccgttatcaacttgaaaaagtggcaccgagtcggtgcttaacaaagcaccagtggtctagtggtagaatagtaccctgccacggtacagacccgggttcgattcccggctggtgcaactgcagtgcgtgctgcgcgttttagagctatgctgttttttttt

SEQ ID NO: 27 - DNA, 480 bp, Artificial Sequence
  Streptococcus pyogenes, Oryza sativa
gggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggcaactgcagtgcgtgctgcgcgttttagagctagaaatagcaagttaaaataaggctagtccgttatcaacttgaaaaagtggcaccgagtcggtgcttttttttt

SEQ ID NO: 28 - DNA, 31 bp, Artificial Sequence; identical to SEQ ID NO: 23
  Streptococcus pyogenes

SEQ ID NO: 29 - DNA, 80 bp, Artificial Sequence
  Streptococcus pyogenes
gcaccacagcatagcaagttaaaataaggctagtccgttatcaacttgaaaaagtggcaccgagtcggtgcttttttttt

SEQ ID NO: 30 - DNA, 31 bp, Artificial Sequence
  Streptococcus pyogenes
gttttagagctacgccgggggcttttttttt

SEQ ID NO: 31 - DNA, 80 bp, Artificial Sequence
  Streptococcus pyogenes
ggcccccggcgtagcaagttaaaataaggctagtccgttatcaacttgaaaaagtggcaccgagtcggtgcttttttttt

SEQ ID NO: 32 - DNA, 61 bp, Artificial Sequence
  Streptococcus pyogenes
gttttagagctatgctgttttggagctcgagctccccggggagctcgagctcttttttttt

SEQ ID NO: 33 - DNA, 109 bp, Artificial Sequence
  Streptococcus pyogenes
gagctcgagctccccggggagctcgagctccaaaacagcatagcaagttaaaataaggctagtccgttatcaacttgaaaaagtggcaccgagtcggtgcttttttttt

SEQ ID NO: 34 - DNA, 65 bp, Artificial Sequence
  Streptococcus pyogenes
gttttagagctatgctgttttggagctcgagctccccgaaaggggagctcgagctcttttttttt

SEQ ID NO: 35 - DNA, 113 bp, Artificial Sequence
  Streptococcus pyogenes
gagctcgagctcccctttcggggagctcgagctccaaaacagcatagcaagttaaaataaggctagtccgttatcaacttgaaaaagtggcaccgagtcggtgcttttttttt

SEQ ID NO: 36 - DNA, 1866 bp, Artificial Sequence
  Streptococcus pyogenes, Wheat Dwarf Virus
atgaagaggccatggtagtgaacagaagtccggcaggtccttagcgaaaaaacggggtgtgccagaaaactctatgctctaccctgcgtggaggtgtgaattctgcacactgcaaatgcaatgtgtccaatgctttatatagggcaggttttggcgggagaacagggcccttgtgttcccacgggagcgtagcgtatcgtgtgggccctgttcggtgtgtggtcggggggcctccacgcgggttataatattaccccgcgtggtggcccccgacgcgcactcggcttttcgtgagtgcgcggaggcttttggaccacatcttttctgatcactttcgtggaagatgttgatttatcacacttttgacttggaaatctgtgccatgccttagcttataaggaagtgcgcggtagcccatctcgatggagcaggcaatagcccccccgcttcctatacgggactatcaataccagaccccttgggaccctttgtgaaagttgaattacggcatagccgaaggaataacagaatcgtttcacactttcgtaacaaaggtcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagcttgactgcagtgcgtgctgcgcgttttagagctatgctgtggtgttttttttttttgtgaaagttgaattacggcatagccgaaggaataacagaatcgtttcacactttcgtaacaaaggtcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagcttgcaccacagcatagcaagttaaaataaggctagtccgttatcaacttgaaaaagtggcaccgagtcggtgctttttttttcctaggacgcgtcgtgttatttcaaagccatcggcattcagtaataaaataatattttatttatctcatgtcattcgattacagaggctcggctacgagcaaagacaaaccaaattataacaaaaaacaacccttacacaatgacatcggaaaacgaaatacaacaccctgagatattacatttatagaaactgtacgccgtccgcgctaggacag

SEQ ID NO: 37 - DNA, 932 bp, Artificial Sequence
  Streptococcus pyogenes, Oryza sativa
aaggaatctttaaacatacgaacagatcacttaaagttcttctgaagcaacttaaagttatcaggcatgcatggatcttggaggaatcagatgtgcagtcagggaccatagcacaagacaggcgtcttctactggtgctaccagcaaatgctggaagccgggaacactgggtacgttggaaaccacgtgatgtgaagaagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctgatttaaaagagttgtgcagatgatccgtggcaacaaagcaccagtggtctagtggtagaatagtaccctgccacggtacagacccgggttcgattcccggctggtgcaggaaccattcaaaacagcatagcaagttaaaataaggctagtccgttatcaacttgaaaaagtggcaccgagtcggtgcttaacaaagcaccagtggtctagtggtagaatagtaccctgccacggtacagacccgggttcgattcccggctggtgcaactgcagtgcgtgctgcgcgttttagagctatgctgtaacaaagcaccagtggtctagtggtagaatagtaccctgccacggtacagacccgggttcgattcccggctggtgcaggaaccattcaaaacagcatagcaagttaaaataaggctagtccgttatcaacttgaaaaagtggcaccgagtcggtgcttaacaaagcaccagtggtctagtggtagaatagtaccctgccacggtacagacccgggttcgattcccggctggtgcaactgcagtgcgtgctgcgcgttttagagctatgctgttttttttt

SEQ ID NO: 38 - DNA, 916 bp, Artificial Sequence
  Streptococcus pyogenes, Oryza sativa
aaggaatctttaaacatacgaacagatcacttaaagttcttctgaagcaacttaaagttatcaggcatgcatggatcttggaggaatcagatgtgcagtcagggaccatagcacaagacaggcgtcttctactggtgctaccagcaaatgctggaagccgggaacactgggtacgttggaaaccacgtgatgtgaagaagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctgatttaaaagagttgtgcagatgatccgtggcaacaaagcaccagtggtctagtggtagaatagtaccctgccacggtacagacccgggttcgattcccggctggtgcagcaaaacagcatagcaagttaaaataaggctagtccgttatcaacttgaaaaagtggcaccgagtcggtgcttaacaaagcaccagtggtctagtggtagaatagtaccctgccacggtacagacccgggttcgattcccggctggtgcaactgcagtgcgtgctgcgcgttttagagctatgctgtaacaaagcaccagtggtctagtggtagaatagtaccctgccacggtacagacccgggttcgattcccggctggtgcagcaaaacagcatagcaagttaaaataaggctagtccgttatcaacttgaaaaagtggcaccgagtcggtgcttaacaaagcaccagtggtctagtggtagaatagtaccctgccacggtacagacccgggttcgattcccggctggtgcaactgcagtgcgtgctgcgcgttttagagctatgctgttttttttt

SEQ ID NO: 39 - protein, 1389 aa, Artificial Sequence
  Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELVGITIMERSSFEKNPVDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSSPPKKKRKVSWKDASGWSRM

SEQ ID NO: 40 - DNA, 2683 bp, Artificial Sequence
  Streptococcus pyogenes, Oryza sativa, Saccharum officinarum, Agrobacterium tumefaciens
cattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcaggtaacaaagcaccagtggtctagtggtagaatagtaccctgccacggtacagacccgggttcgattcccggctggtgcaggaaccattcaaaacagcatagcaagttaaaataaggctagtccgttatcaacttgaaaaagtggcaccgagtcggtgctttaacaaagcaccagtggtctagtggtagaatagtaccctgccacggtacagacccgggttcgattcccggctggtgcaactgcagtgcgtgctgcgcgttttagagctatgctgttttgaacaaagcaccagtggtctagtggtagaatagtaccctgccacggtacagacccgggttcgattcccggctggtgcaggaaccattcaaaacagcatagcaagttaaaataaggctagtccgttatcaacttgaaaaagtggcaccgagtcggtgctttaacaaagcaccagtggtctagtggtagaatagtaccctgccacggtacagacccgggttcgattcccggctggtgcaactgcagtgcgtgctgcgcgttttagagctatgctgttttgaacaaagcaccagtggtctagtggtagaatagtaccctgccacggtacagacccgggttcgattcccggctggtgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 41 - DNA, 916 bp, Artificial Sequence; identical to SEQ ID NO: 38
  Streptococcus pyogenes, Oryza sativa, Arabidopsis thaliana

SEQ ID NO: 42 - DNA, 2667 bp, Artificial Sequence
  Streptococcus pyogenes, Oryza sativa, Saccharum officinarum, Agrobacterium tumefaciens
cattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcaggtaacaaagcaccagtggtctagtggtagaatagtaccctgccacggtacagacccgggttcgattcccggctggtgcagcaaaacagcatagcaagttaaaataaggctagtccgttatcaacttgaaaaagtggcaccgagtcggtgctttaacaaagcaccagtggtctagtggtagaatagtaccctgccacggtacagacccgggttcgattcccggctggtgcaactgcagtgcgtgctgcgcgttttagagctatgctgttttgaacaaagcaccagtggtctagtggtagaatagtaccctgccacggtacagacccgggttcgattcccggctggtgcagcaaaacagcatagcaagttaaaataaggctagtccgttatcaacttgaaaaagtggcaccgagtcggtgctttaacaaagcaccagtggtctagtggtagaatagtaccctgccacggtacagacccgggttcgattcccggctggtgcaactgcagtgcgtgctgcgcgttttagagctatgctgttttgaacaaagcaccagtggtctagtggtagaatagtaccctgccacggtacagacccgggttcgattcccggctggtgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 43 - DNA, 925 bp, Artificial Sequence
  Streptococcus pyogenes, Oryza sativa
aaggaatctttaaacatacgaacagatcacttaaagttcttctgaagcaacttaaagttatcaggcatgcatggatcttggaggaatcagatgtgcagtcagggaccatagcacaagacaggcgtcttctactggtgctaccagcaaatgctggaagccgggaacactgggtacgttggaaaccacgtgatgtgaagaagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctgatttaaaagagttgtgcagatgatccgtggcaacaaagcaccagtggtctagtggtagaatagtaccctgccacggtacagacccgggttcgattcccggctggtgcagcaccacagcatagcaagttaaaataaggctagtccgttatcaacttgaaaaagtggcaccgagtcggtgcttaacaaagcaccagtggtctagtggtagaatagtaccctgccacggtacagacccgggttcgattcccggctggtgcaactgcagtgcgtgctgcgcgttttagagctatgctgtggtgaacaaagcaccagtggtctagtggtagaatagtaccctgccacggtacagacccgggttcgattcccggctggtgcagcaccacagcatagcaagttaaaataaggctagtccgttatcaacttgaaaaagtggcaccgagtcggtgcttaacaaagcaccagtggtctagtggtagaatagtaccctgccacggtacagacccgggttcgattcccggctggtgcaactgcagtgcgtgctgcgcgttttagagctatgctgtggtgttttttttt

SEQ ID NO: 44 - DNA, 2667 bp, Artificial Sequence
  Streptococcus pyogenes, Oryza sativa, Saccharum officinarum, Agrobacterium tumefaciens
cattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcaggtaacaaagcaccagtggtctagtggtagaatagtaccctgccacggtacagacccgggttcgattcccggctggtgcagcaccacagcatagcaagttaaaataaggctagtccgttatcaacttgaaaaagtggcaccgagtcggtgctttaacaaagcaccagtggtctagtggtagaatagtaccctgccacggtacagacccgggttcgattcccggctggtgcaactgcagtgcgtgctgcgcgttttagagctatgctgtggtgaacaaagcaccagtggtctagtggtagaatagtaccctgccacggtacagacccgggttcgattcccggctggtgcagcaccacagcatagcaagttaaaataaggctagtccgttatcaacttgaaaaagtggcaccgagtcggtgctttaacaaagcaccagtggtctagtggtagaatagtaccctgccacggtacagacccgggttcgattcccggctggtgcaactgcagtgcgtgctgcgcgttttagagctatgctgtggtgaacaaagcaccagtggtctagtggtagaatagtaccctgccacggtacagacccgggttcgattcccggctggtgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 45 - DNA, 19 bp, Artificial Sequence
  Oryza sativa
actgcagtgcgtgctgcgc

SEQ ID NO: 46 - DNA, 19 bp, Artificial Sequence
  Escherichia coli
ccctatccccgttgacgac

SEQ ID NO: 47 - DNA, 23 bp, Artificial Sequence
  Escherichia coli
ctgatagtggtctccttgtcgct

SEQ ID NO: 48 - DNA, 23 bp, Artificial Sequence
  Escherichia coli
ttcgccttcagcctgcacgacct

SEQ ID NO: 49 - DNA, 19 bp, Artificial Sequence
  Streptococcus pyogenes
ttgtgctgctccacgaaca

SEQ ID NO: 50 - DNA, 20 bp, Artificial Sequence
  Streptococcus pyogenes
gccagccactacgagaagct

SEQ ID NO: 51 - DNA, 24 bp, Artificial Sequence
  Streptococcus pyogenes
ctgcttctgctcgttgtcctccgg

SEQ ID NO: 52 - DNA, 17 bp, Oryza sativa
tgcgacgagccatgctg

SEQ ID NO: 53 - DNA, 23 bp, Oryza sativa
gcagtctggactacagcatgacc

SEQ ID NO: 54 - DNA, 13 bp, Oryza sativa
cagcgcagcacgc

SEQ ID NO: 55 - RNA, 22 nt, Artificial Sequence
  Streptococcus pyogenes
guuuuagagcuaugcuguuuug

SEQ ID NO: 56 - RNA, 88 nt, Artificial Sequence
  Streptococcus pyogenes
ggaaccauucaaaacagcauagcaaguuaaaauaaggcuaguccguuaucaacuugaaaaaguggcaccgagucggugcuuuuuuuuu

SEQ ID NO: 57 - RNA, 22 nt, Artificial Sequence
  Streptococcus pyogenes
gguuuagagcuaugcuguuuug

SEQ ID NO: 58 - RNA, 88 nt, Artificial Sequence
  Streptococcus pyogenes
ggaaccauucaaaacagcauagcaaguuaaacuaaggcuaguccguuaucaacuugaaaaaguggcaccgagucggugcuuuuuuuuu

SEQ ID NO: 59 - RNA, 22 nt, Artificial Sequence
  Streptococcus pyogenes
guguuagagcuaugcuguuuug

SEQ ID NO: 60 - RNA, 88 nt, Artificial Sequence
  Streptococcus pyogenes
ggaaccauucaaaacagcauagcaaguuaacauaaggcuaguccguuaucaacuugaaaaaguggcaccgagucggugcuuuuuuuuu

SEQ ID NO: 61 - RNA, 22 nt, Artificial Sequence
  Streptococcus pyogenes
guuguagagcuaugcuguuuug

SEQ ID NO: 62 - RNA, 88 nt, Artificial Sequence
  Streptococcus pyogenes
ggaaccauucaaaacagcauagcaaguuacaauaaggcuaguccguuaucaacuugaaaaaguggcaccgagucggugcuuuuuuuuu

SEQ ID NO: 63 - RNA, 22 nt, Artificial Sequence
  Streptococcus pyogenes
guuugagagcuaugcuguuuug

SEQ ID NO: 64 - RNA, 88 nt, Artificial Sequence
  Streptococcus pyogenes
ggaaccauucaaaacagcauagcaaguucaaauaaggcuaguccguuaucaacuugaaaaaguggcaccgagucggugcuuuuuuuuu

SEQ ID NO: 65 - RNA, 22 nt, Artificial Sequence
  Streptococcus pyogenes
guuuaagagcuaugcuguuuug

SEQ ID NO: 66 - RNA, 88 nt, Artificial Sequence
  Streptococcus pyogenes
ggaaccauucaaaacagcauagcaaguuuaaauaaggcuaguccguuaucaacuugaaaaaguggcaccgagucggugcuuuuuuuuu

SEQ ID NO: 67 - RNA, 22 nt, Artificial Sequence
  Streptococcus pyogenes
guuucagagcuaugcuguuuug

SEQ ID NO: 68 - RNA, 88 nt, Artificial Sequence
  Streptococcus pyogenes
ggaaccauucaaaacagcauagcaaguugaaauaaggcuaguccguuaucaacuugaaaaaguggcaccgagucggugcuuuuuuuuu

SEQ ID NO: 69 - RNA, 40 nt, Artificial Sequence
  Streptococcus pyogenes
guuuuagagcuaugcuguuuugaaugguuccuuuuuuuuu

SEQ ID NO: 70 - RNA, 88 nt, Artificial Sequence; identical to SEQ ID NO: 56
  Streptococcus pyogenes

SEQ ID NO: 71 - RNA, 22 nt, Artificial Sequence
  Streptococcus pyogenes
guuuuagagcuaugcuguuuug

SEQ ID NO: 72 - RNA, 80 nt, Artificial Sequence
  Streptococcus pyogenes
gcaaaacagcauagcaaguuaaaauaaggcuaguccguuaucaacuugaaaaaguggcaccgagucggugcuuuuuuuuu

SEQ ID NO: 73 - RNA, 52 nt, Artificial Sequence
  Streptococcus pyogenes
guuuuagagcuaugcuguuuugagcucggguaggcugucaaccuaccgccgu

SEQ ID NO: 74 - RNA, 126 nt, Artificial Sequence
  Streptococcus pyogenes
gcgguccccuuauugccugacaagcugagggccacccuggaaccauucaaaacagcauagcaaguuaaaauaaggcuaguccguuaucaacuugaaaaaguggcaccgagucggugcuuuuuuuuu

SEQ ID NO: 75 - RNA, 22 nt, Artificial Sequence
  Streptococcus pyogenes
guuuuagagcuaugcuguggug

SEQ ID NO: 76 - RNA, 88 nt, Artificial Sequence
  Streptococcus pyogenes
ggaaccauucaccacagcauagcaaguuaaaauaaggcuaguccguuaucaacuugaaaaaguggcaccgagucggugcuuuuuuuuu

SEQ ID NO: 77 - RNA, 22 nt, Artificial Sequence
  Streptococcus pyogenes
guuuuagagcuaugcuguggug

SEQ ID NO: 78 - RNA, 80 nt, Artificial Sequence
  Streptococcus pyogenes
gcaccacagcauagcaaguuaaaauaaggcuaguccguuaucaacuugaaaaaguggcaccgagucggugcuuuuuuuuu

SEQ ID NO: 79 - RNA, 22 nt, Artificial Sequence
  Streptococcus pyogenes
guuuuagagcuacgccgggggc

SEQ ID NO: 80 - RNA, 80 nt, Artificial Sequence
  Streptococcus pyogenes
ggcccccggcguagcaaguuaaaauaaggcuaguccguuaucaacuugaaaaaguggcaccgagucggugcuuuuuuuuu

SEQ ID NO: 81 - RNA, 52 nt, Artificial Sequence
  Streptococcus pyogenes
guuuuagagcuaugcuguuuuggagcucgagcuccccggggagcucgagcuc

SEQ ID NO: 82 - RNA, 109 nt, Artificial Sequence
  Streptococcus pyogenes
gagcucgagcuccccggggagcucgagcuccaaaacagcauagcaaguuaaaauaaggcuaguccguuaucaacuugaaaaaguggcaccgagucggugcuuuuuuuuu

SEQ ID NO: 83 - RNA, 56 nt, Artificial Sequence
  Streptococcus pyogenes
guuuuagagcuaugcuguuuuggagcucgagcuccccgaaaggggagcucgagcuc

SEQ ID NO: 84 - RNA, 113 nt, Artificial Sequence
  Streptococcus pyogenes
gagcucgagcuccccuuucggggagcucgagcuccaaaacagcauagcaaguuaaaauaaggcuaguccguuaucaacuugaaaaaguggcaccgagucggugcuuuuuuuuu

SEQ ID NO: 85 - DNA, 1797 bp, Artificial Sequence
  Saccharum officinarum
cattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcag

SEQ ID NO: 86 - DNA, 380 bp, Artificial Sequence
  Oryza sativa
aaggaatctttaaacatacgaacagatcacttaaagttcttctgaagcaacttaaagttatcaggcatgcatggatcttggaggaatcagatgtgcagtcagggaccatagcacaagacaggcgtcttctactggtgctaccagcaaatgctggaagccgggaacactgggtacgttggaaaccacgtgatgtgaagaagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctgatttaaaagagttgtgcagatgatccgtggc

SEQ ID NO: 87 - DNA, 664 bp, Artificial Sequence
  Streptococcus pyogenes, Oryza sativa
  misc_feature (458)..(477): n is a, c, g, or t
aaggaatctttaaacatacgaacagatcacttaaagttcttctgaagcaacttaaagttatcaggcatgcatggatcttggaggaatcagatgtgcagtcagggaccatagcacaagacaggcgtcttctactggtgctaccagcaaatgctggaagccgggaacactgggtacgttggaaaccacgtgatgtgaagaagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctgatttaaaagagttgtgcagatgatccgtggcaacaaagcaccagtggtctagtggtagaatagtaccctgccacggtacagacccgggttcgattcccggctggtgcannnnnnnnnnnnnnnnnnnngttttagagctatgctgttttgaacaaagcaccagtggtctagtggtagaatagtaccctgccacggtacagacccgggttcgattcccggctggtgcaggaaccattcaaaacagcatagcaagttaaaataaggctagtccgttatcaacttgaaaaagtggcaccgagtcggtgcttttttttt

SEQ ID NO: 88 - DNA, 661 bp, Artificial Sequence
  Streptococcus pyogenes, Oryza sativa,
  misc_feature (616)..(635): n is a, c, g, or t
aaggaatctttaaacatacgaacagatcacttaaagttcttctgaagcaacttaaagttatcaggcatgcatggatcttggaggaatcagatgtgcagtcagggaccatagcacaagacaggcgtcttctactggtgctaccagcaaatgctggaagccgggaacactgggtacgttggaaaccacgtgatgtgaagaagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctgatttaaaagagttgtgcagatgatccgtggcaacaaagcaccagtggtctagtggtagaatagtaccctgccacggtacagacccgggttcgattcccggctggtgcaggaaccattcaaaacagcatagcaagttaaaataaggctagtccgttatcaacttgaaaaagtggcaccgagtcggtgcttaacaaagcaccagtggtctagtggtagaatagtaccctgccacggtacagacccgggttcgattcccggctggtgcannnnnnnnnnnnnnnnnnnngttttagagctatgctgttttttttt

SEQ ID NO: 89 - DNA, 934 bp, Artificial Sequence
  Streptococcus pyogenes, Oryza sativa
  misc_feature (616)..(635): n is a, c, g, or t
  misc_feature (889)..(908): n is a, c, g, or t
aaggaatctttaaacatacgaacagatcacttaaagttcttctgaagcaacttaaagttatcaggcatgcatggatcttggaggaatcagatgtgcagtcagggaccatagcacaagacaggcgtcttctactggtgctaccagcaaatgctggaagccgggaacactgggtacgttggaaaccacgtgatgtgaagaagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctgatttaaaagagttgtgcagatgatccgtggcaacaaagcaccagtggtctagtggtagaatagtaccctgccacggtacagacccgggttcgattcccggctggtgcaggaaccattcaaaacagcatagcaagttaaaataaggctagtccgttatcaacttgaaaaagtggcaccgagtcggtgcttaacaaagcaccagtggtctagtggtagaatagtaccctgccacggtacagacccgggttcgattcccggctggtgcannnnnnnnnnnnnnnnnnnngttttagagctatgctgtaacaaagcaccagtggtctagtggtagaatagtaccctgccacggtacagacccgggttcgattcccggctggtgcaggaaccattcaaaacagcatagcaagttaaaataaggctagtccgttatcaacttgaaaaagtggcaccgagtcggtgcttaacaaagcaccagtggtctagtggtagaatagtaccctgccacggtacagacccgggttcgattcccggctggtgcannnnnnnnnnnnnnnnnnnngttttagagctatgctgttttttttt

SEQ ID NO: 90 - DNA, 918 bp, Artificial Sequence
  Streptococcus pyogenes, Oryza sativa, Arabidopsis thaliana
  misc_feature (608)..(627): n is a, c, g, or t
  misc_feature (873)..(892): n is a, c, g, or t
aaggaatctttaaacatacgaacagatcacttaaagttcttctgaagcaacttaaagttatcaggcatgcatggatcttggaggaatcagatgtgcagtcagggaccatagcacaagacaggcgtcttctactggtgctaccagcaaatgctggaagccgggaacactgggtacgttggaaaccacgtgatgtgaagaagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctgatttaaaagagttgtgcagatgatccgtggcaacaaagcaccagtggtctagtggtagaatagtaccctgccacggtacagacccgggttcgattcccggctggtgcagcaaaacagcatagcaagttaaaataaggctagtccgttatcaacttgaaaaagtggcaccgagtcggtgcttaacaaagcaccagtggtctagtggtagaatagtaccctgccacggtacagacccgggttcgattcccggctggtgcannnnnnnnnnnnnnnnnnnngttttagagctatgctgtaacaaagcaccagtggtctagtggtagaatagtaccctgccacggtacagacccgggttcgattcccggctggtgcagcaaaacagcatagcaagttaaaataaggctagtccgttatcaacttgaaaaagtggcaccgagtcggtgcttaacaaagcaccagtggtctagtggtagaatagtaccctgccacggtacagacccgggttcgattcccggctggtgcannnnnnnnnnnnnnnnnnnngttttagagctatgctgttttttttt

SEQ ID NO: 91 - DNA, 2685 bp, Artificial Sequence
  Streptococcus pyogenes, Oryza sativa, Agrobacterium tumefaciens, Saccharum officinarum
  misc_feature (2036)..(2055): n is a, c, g, or t
  misc_feature (2314)..(2333): n is a, c, g, or t
cattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcaggtaacaaagcaccagtggtctagtggtagaatagtaccctgccacggtacagacccgggttcgattcccggctggtgcaggaaccattcaaaacagcatagcaagttaaaataaggctagtccgttatcaacttgaaaaagtggcaccgagtcggtgctttaacaaagcaccagtggtctagtggtagaatagtaccctgccacggtacagacccgggttcgattcccggctggtgcannnnnnnnnnnnnnnnnnnngttttagagctatgctgttttgaacaaagcaccagtggtctagtggtagaatagtaccctgccacggtacagacccgggttcgattcccggctggtgcaggaaccattcaaaacagcatagcaagttaaaataaggctagtccgttatcaacttgaaaaagtggcaccgagtcggtgctttaacaaagcaccagtggtctagtggtagaatagtaccctgccacggtacagacccgggttcgattcccggctggtgcannnnnnnnnnnnnnnnnnnngttttagagctatgctgttttgaacaaagcaccagtggtctagtggtagaatagtaccctgccacggtacagacccgggttcgattcccggctggtgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 92 - DNA, 918 bp, Artificial Sequence; identical to SEQ ID NO: 90
  Streptococcus pyogenes, Oryza sativa, Arabidopsis thaliana
  misc_feature (608)..(627): n is a, c, g, or t
  misc_feature (873)..(892): n is a, c, g, or t

SEQ ID NO: 93 - DNA, 2669 bp, Artificial Sequence
  Streptococcus pyogenes, Saccharum officinarum, Agrobacterium tumefaciens
  misc_feature (2028)..(2047): n is a, c, g, or t
  misc_feature (2298)..(2317): n is a, c, g, or t
cattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcaggtaacaaagcaccagtggtctagtggtagaatagtaccctgccacggtacagacccgggttcgattcccggctggtgcagcaaaacagcatagcaagttaaaataaggctagtccgttatcaacttgaaaaagtggcaccgagtcggtgctttaacaaagcaccagtggtctagtggtagaatagtaccctgccacggtacagacccgggttcgattcccggctggtgcannnnnnnnnnnnnnnnnnnngttttagagctatgctgttttgaacaaagcaccagtggtctagtggtagaatagtaccctgccacggtacagacccgggttcgattcccggctggtgcagcaaaacagcatagcaagttaaaataaggctagtccgttatcaacttgaaaaagtggcaccgagtcggtgctttaacaaagcaccagtggtctagtggtagaatagtaccctgccacggtacagacccgggttcgattcccggctggtgcannnnnnnnnnnnnnnnnnnngttttagagctatgctgttttgaacaaagcaccagtggtctagtggtagaatagtaccctgccacggtacagacccgggttcgattcccggctggtgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 94 - DNA, 927 bp, Artificial Sequence
  Streptococcus pyogenes, Oryza sativa
  misc_feature (608)..(627): n is a, c, g, or t
  misc_feature (877)..(896): n is a, c, g, or t
aaggaatctttaaacatacgaacagatcacttaaagttcttctgaagcaacttaaagttatcaggcatgcatggatcttggaggaatcagatgtgcagtcagggaccatagcacaagacaggcgtcttctactggtgctaccagcaaatgctggaagccgggaacactgggtacgttggaaaccacgtgatgtgaagaagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctgatttaaaagagttgtgcagatgatccgtggcaacaaagcaccagtggtctagtggtagaatagtaccctgccacggtacagacccgggttcgattcccggctggtgcagcaccacagcatagcaagttaaaataaggctagtccgttatcaacttgaaaaagtggcaccgagtcggtgcttaacaaagcaccagtggtctagtggtagaatagtaccctgccacggtacagacccgggttcgattcccggctggtgcannnnnnnnnnnnnnnnnnnngttttagagctatgctgtggtgaacaaagcaccagtggtctagtggtagaatagtaccctgccacggtacagacccgggttcgattcccggctggtgcagcaccacagcatagcaagttaaaataaggctagtccgttatcaacttgaaaaagtggcaccgagtcggtgcttaacaaagcaccagtggtctagtggtagaatagtaccctgccacggtacagacccgggttcgattcccggctggtgcannnnnnnnnnnnnnnnnnnngttttagagctatgctgtggtgttttttttt

SEQ ID NO: 95 - DNA, 2669 bp, Artificial Sequence
  Streptococcus pyogenes, Saccharum officinarum, Agrobacterium tumefaciens
  misc_feature (2028)..(2047): n is a, c, g, or t
  misc_feature (2298)..(2317): n is a, c, g, or t
cattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcaggtaacaaagcaccagtggtctagtggtagaatagtaccctgccacggtacagacccgggttcgattcccggctggtgcagcaccacagcatagcaagttaaaataaggctagtccgttatcaacttgaaaaagtggcaccgagtcggtgctttaacaaagcaccagtggtctagtggtagaatagtaccctgccacggtacagacccgggttcgattcccggctggtgcannnnnnnnnnnnnnnnnnnngttttagagctatgctgtggtgaacaaagcaccagtggtctagtggtagaatagtaccctgccacggtacagacccgggttcgattcccggctggtgcagcaccacagcatagcaagttaaaataaggctagtccgttatcaacttgaaaaagtggcaccgagtcggtgctttaacaaagcaccagtggtctagtggtagaatagtaccctgccacggtacagacccgggttcgattcccggctggtgcannnnnnnnnnnnnnnnnnnngttttagagctatgctgtggtgaacaaagcaccagtggtctagtggtagaatagtaccctgccacggtacagacccgggttcgattcccggctggtgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 96 - RNA, 32 nt, Artificial Sequence
  Streptococcus pyogenes
ggaaccauucaaaacagcauagcaaguuaaaa

SEQ ID NO: 97 - RNA, 32 nt, Artificial Sequence
  Streptococcus pyogenes
ggaaccauucaaaacagcauagcaaguuaaac

SEQ ID NO: 98 - RNA, 32 nt, Artificial Sequence
  Streptococcus pyogenes
ggaaccauucaaaacagcauagcaaguuaaca

SEQ ID NO: 99 - RNA, 32 nt, Artificial Sequence
  Streptococcus pyogenes
ggaaccauucaaaacagcauagcaaguuacaa

SEQ ID NO: 100 - RNA, 32 nt, Artificial Sequence
  Streptococcus pyogenes
ggaaccauucaaaacagcauagcaaguucaaa

SEQ ID NO: 101 - RNA, 32 nt, Artificial Sequence
  Streptococcus pyogenes
ggaaccauucaaaacagcauagcaaguuuaaa

SEQ ID NO: 102 - RNA, 32 nt, Artificial Sequence
  Streptococcus pyogenes
ggaaccauucaaaacagcauagcaaguugaaa

SEQ ID NO: 103 - RNA, 32 nt, Artificial Sequence; identical to SEQ ID NO: 96
  Streptococcus pyogenes

SEQ ID NO: 104 - RNA, 24 nt, Artificial Sequence
  Streptococcus pyogenes
gcaaaacagcauagcaaguuaaaa

SEQ ID NO: 105 - RNA, 70 nt, Artificial Sequence
  Streptococcus pyogenes
gcgguccccuuauugccugacaagcugagggccacccuggaaccauucaaaacagcauagcaaguuaaaa

SEQ ID NO: 106 - RNA, 32 nt, Artificial Sequence
  Streptococcus pyogenes
ggaaccauucaccacagcauagcaaguuaaaa

SEQ ID NO: 107 - RNA, 24 nt, Artificial Sequence
  Streptococcus pyogenes
gcaccacagcauagcaaguuaaaa

SEQ ID NO: 108 - RNA, 24 nt, Artificial Sequence
  Streptococcus pyogenes
ggcccccggcguagcaaguuaaaa

SEQ ID NO: 109 - RNA, 53 nt, Artificial Sequence
  Streptococcus pyogenes
gagcucgagcuccccggggagcucgagcuccaaaacagcauagcaaguuaaaa

SEQ ID NO: 110 - RNA, 57 nt, Artificial Sequence
  Streptococcus pyogenes
gagcucgagcuccccuuucggggagcucgagcuccaaaacagcauagcaaguuaaaa

SEQ ID NO: 111 - DNA, 4170 bp, Artificial Sequence
  Streptococcus pyogenes
atggacaagaagtacagcatcggcctggacatcggcaccaacagcgtgggctgggccgtgatcaccgacgagtacaaggtgccgagcaagaagttcaaggtgctgggcaacaccgacaggcacagcatcaagaagaacctgatcggcgccctgctgttcgacagcggcgagaccgccgaggccaccaggctgaagaggaccgccaggaggaggtacaccaggaggaagaacaggatctgctacctgcaggagatcttcagcaacgagatggccaaggtggacgacagcttcttccacaggctggaggagagcttcctggtggaggaggacaagaagcacgagaggcacccgatcttcggcaacatcgtggacgaggtggcctaccacgagaagtacccgaccatctaccacctgaggaagaagctggtggacagcaccgacaaggccgacctgaggctgatctacctggccctggcccacatgatcaagttcaggggccacttcctgatcgagggcgacctgaacccggacaacagcgacgtggacaagctgttcatccagctggtgcagacctacaaccagctgttcgaggagaacccgatcaacgccagcggcgtggacgccaaggccatcctgagcgccaggctgagcaagagcaggaggctggagaacctgatcgcccagctgccgggcgagaagaagaacggcctgttcggcaacctgatcgccctgagcctgggcctgaccccgaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgagggtgaacaccgagatcaccaaggccccgctgagcgccagcatgatcaagaggtacgacgagcaccaccaggacctgaccctgctgaaggccctggtgaggcagcagctgccggagaagtacaaggagatcttcttcgaccagagcaagaacggctacgccggctacatcgacggcggcgccagccaggaggagttctacaagttcatcaagccgatcctggagaagatggacggcaccgaggagctgctggtgaagctgaacagggaggacctgctgaggaagcagaggaccttcgacaacggcagcatcccgcaccagatccacctgggcgagctgcacgccatcctgaggaggcaggaggacttctacccgttcctgaaggacaacagggagaagatcgagaagatcctgaccttccgcatcccgtactacgtgggcccgctggccaggggcaacagcaggttcgcctggatgaccaggaagagcgaggagaccatcaccccgtggaacttcgaggaggtggtggacaagggcgccagcgcccagagcttcatcgagaggatgaccaacttcgacaagaacctgccgaacgagaaggtgctgccgaagcacagcctgctgtacgagtacttcaccgtgtacaacgagctgaccaaggtgaagtacgtgaccgagggcatgaggaagccggccttcctgagcggcgagcagaagaaggccatcgtggacctgctgttcaagaccaacaggaaggtgaccgtgaagcagctgaaggaggactacttcaagaagatcgagtgcttcgacagcgtggagatcagcggcgtggaggacaggttcaacgccagcctgggcacctaccacgacctgctgaagatcatcaaggacaaggacttcctggacaacgaggagaacgaggacatcctggaggacatcgtgctgaccctgaccctgttcgaggacagggagatgatcgaggagaggctgaagacctacgcccacctgttcgacgacaaggtgatgaagcagctgaagaggaggaggtacaccggctggggcaggctgagcaggaagctgatcaacggcatcagggacaagcagagcggcaagaccatcctggacttcctgaagagcgacggcttcgccaacaggaacttcatgcagctgatccacgacgacagcctgaccttcaaggaggacatccagaaggcccaggtgagcggccagggcgacagcctgcacgagcacatcgccaacctggccggcagcccggccatcaagaagggcatcctgcagaccgtgaaggtggtggacgagctggtgaaggtgatgggcaggcacaagccggagaacatcgtgatcgagatggccagggagaaccagaccacccagaagggccagaagaacagcagggagaggatgaagaggatcgaggagggcatcaaggagctgggcagccagatcctgaaggagcacccggtggagaacacccagctgcagaacgagaagctgtacctgtactacctgcagaacggcagggacatgtacgtggaccaggagctggacatcaacaggctgagcgactacgacgtggaccacatcgtgccgcagagcttcctgaaggacgacagcatcgacaacaaggtgctgaccaggagcgacaagaacaggggcaagagcgacaacgtgccgagcgaggaggtggtgaagaagatgaaaaactactggaggcagctgctgaacgccaagctgatcacccagaggaagttcgacaacctgaccaaggccgagaggggcggcctgagcgagctggacaaggccggcttcattaaaaggcagctggtggagaccaggcagatcaccaagcacgtggcccagatcctggacagcaggatgaacaccaagtacgacgagaacgacaagctgatcagggaggtgaaggtgatcaccctgaagagcaagctggtgagcgacttcaggaaggacttccagttctacaaggtgagggagatcaataattaccaccacgcccacgacgcctacctgaacgccgtggtgggcaccgccctgattaaaaagtacccgaagctggagagcgagttcgtgtacggcgactacaaggtgtacgacgtgaggaagatgatcgccaagagcgagcaggagatcggcaaggccaccgccaagtacttcttctacagcaacatcatgaacttcttcaagaccgagatcaccctggccaacggcgagatcaggaagaggccgctgatcgagaccaacggcgagaccggcgagatcgtgtgggacaagggcagggacttcgccaccgtgaggaaggtgctgtccatgccgcaggtgaacatcgtgaagaagaccgaggtgcagaccggcggcttcagcaaggagagcatcctgccgaagaggaacagcgacaagctgatcgccaggaagaaggactgggatccgaagaagtacggcggcttcgacagcccgaccgtggcctacagcgtgctggtggtggccaaggtggagaagggcaagagcaagaagctgaagagcgtgaaggagctggtgggcatcaccatcatggagaggagcagcttcgagaagaacccagtggacttcctggaggccaagggctacaaggaggtgaagaaggacctgatcattaaactgccgaagtacagcctgttcgagctggagaacggcaggaagaggatgctggccagcgccggcgagctgcagaagggcaacgagctggccctgccgagcaagtacgtgaacttcctgtacctggccagccactacgagaagctgaagggcagcccggaggacaacgagcagaagcagctgttcgtggagcagcacaagcactacctggacgagatcatcgagcagatcagcgagttcagcaagagggtgatcctggccgacgccaacctggacaaggtgctgagcgcctacaacaagcacagggacaagccgatcagggagcaggccgagaacatcatccacctgttcaccctgaccaacctgggcgccccggccgccttcaagtacttcgacaccaccatcgacaggaagaggtacaccagcaccaaggaggtgctggacgccaccctgatccaccagagcatcaccggcctgtacgagaccaggatcgacctgagccagctgggcggcgacagcagcccgccgaagaagaagaggaaggtgagctggaaggacgccagcggctggagcaggatgtga

SEQ ID NO: 112 - DNA, 77 bp, Artificial Sequence
  Oryza sativa
aacaaagcaccagtggtctagtggtagaatagtaccctgccacggtacagacccgggttcgattcccggctggtgca

SEQ ID NO: 113 - RNA, 42 nt, Artificial Sequence
  Streptococcus pyogenes
  misc_feature (1)..(20): n is a, c, g, or u
nnnnnnnnnnnnnnnnnnnnguuuuagagcuaugcuguuuug

SEQ ID NO: 114 - RNA, 85 nt, Artificial Sequence
  Streptococcus pyogenes
ggaaccauucaaaacagcauagcaaguuaaaauaaggcuaguccguuaucaacuugaaaaaguggcaccgagucggugcuuuuuu